SEQUENCE LISTING (77 sequences)

SEQ ID NO: 1 - protein, 222 aa, Artificial Sequence
  Spacer LNGFR wild-type long (NWL)
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDN

SEQ ID NO: 2 - DNA, 666 bp, Artificial Sequence
  Spacer LNGFR wild-type long (NWL)
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaat

SEQ ID NO: 3 - protein, 162 aa, Artificial Sequence
  Spacer LNGFR wild-type short (NWS)
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEE

SEQ ID NO: 4 - DNA, 486 bp, Artificial Sequence
  Spacer LNGFR wild-type short (NWS)
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaggaa

SEQ ID NO: 5 - protein, 200 aa, Artificial Sequence
  Spacer LNGFR mutated long (NML)
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEIPGRWITRSTPPEGSDSTAPSTQEPEAPPEQDLIASTVAGVVTTVMGSSQPVVTRGTTDN

SEQ ID NO: 6 - DNA, 600 bp, Artificial Sequence
  Spacer LNGFR mutated long (NML)
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaagccgccagagccgccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaat

SEQ ID NO: 7 - protein, 140 aa, Artificial Sequence
  Spacer LNGFR mutated short (NMS)
KEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEE

SEQ ID NO: 8 - DNA, 420 bp, Artificial Sequence
  Spacer LNGFR mutated short (NMS)
aaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccgcccgggccgccgacgccgagtgcgaggaa

SEQ ID NO: 9 - protein, 34 aa, Homo sapiens
ACPTGLYTHSGECCKACNLGEGVAQPCGANQTVC

SEQ ID NO: 10 - protein, 42 aa, Homo sapiens
PCLDSVTFSDVVSATEPCKPCTECVGLQSMSAPCVEADDAVC

SEQ ID NO: 11 - protein, 39 aa, Homo sapiens
RCAYGYYQDETTGRCEACRVCEAGSGLVFSCQDKQNTVC

SEQ ID NO: 12 - protein, 41 aa, Homo sapiens
ECPDGTYSDEANHVDPCLPCTVCEDTERQLRECTRWADAEC

SEQ ID NO: 13 - protein, 25 aa, Artificial Sequence
  amino acid sequence removed from fourth TNFR-Cys domain
NHVDPCLPCTVCEDTERQLRECTRW

SEQ ID NO: 14 - protein, 427 aa, Homo sapiens
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGANSRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 15 - DNA, 2046 bp, Artificial Sequence
  CD44v6CAR.28z
atggaagcccctgcccagctgctgttcctgctgctgctgtggctgcccgacaccaccggcgagatcgtgctgacacagagccccgccaccctgtctctgagccctggcgagagagccaccctgagctgtagcgccagcagcagcatcaactacatctactggctgcagcagaagcccggccaggcccccagaatcctgatctacctgaccagcaacctggccagcggcgtgcccgccagattttctggcagcggcagcggcaccgacttcaccctgaccatcagcagcctggaacccgaggacttcgccgtgtactactgcctgcagtggtccagcaaccccctgaccttcggcggaggcaccaaggtggaaatcaagcggggtggtggtggttctggtggtggtggttctggcggcggcggctccggtggtggtggatctgaggtgcagctggtggaaagcggcggaggcctggtcaagcctggcggcagcctgagactgagctgtgccgccagcggcttcaccttcagcagctacgacatgagctgggtccgacaggctccaggcaagggactggaatgggtgtccaccatcagcagcggcggcagctacacctactacctggacagcatcaagggccggttcaccatcagccgggacaacgccaagaacagcctgtacctgcagatgaacagcctgcgggccgaggacaccgccgtctactactgtgcccggcagggcctcgactactggggcagaggcaccctggtcaccgtgtccagcggggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - protein, 681 aa, Artificial Sequence
  CD44v6CAR.28z protein
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 261 aa, Artificial Sequence
  CD44v6-specific single-chain fragment (scFV)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSS

SEQ ID NO: 18 - protein, 68 aa, Artificial Sequence
  transmembrane and intracellular co-stimulatory domain
FWVLVVVGGVLACYSLLVTVAFIIFWVRSKRSRLLHSDYMNMTPRRPGPTRKHYQPYAPPRDFAAYRS

SEQ ID NO: 19 - protein, 222 aa, Homo sapiens; identical to SEQ ID NO: 1

SEQ ID NO: 20 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 21 - protein, 669 aa, Artificial Sequence
  Exemplary sequence of a CD44v6CAR.28z, with spacer LNGFR wild-type long (NWL)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 22 - protein, 609 aa, Artificial Sequence
  Exemplary sequence of a CD44v6-CAR28z, with spacer LNGFR wild-type short (NWS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 23 - protein, 647 aa, Artificial Sequence
  Exemplary sequence of a CD44v6-CAR28z, with spacer LNGFR mutated long (NML)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 587 aa, Artificial Sequence
  Exemplary sequence of a CD44v6-CAR28z, with spacer LNGFR mutated short (NMS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 668 aa, Artificial Sequence
  Exemplary sequence of a CD44v6CAR.28z, with spacer LNGFR wild-type long (NWL)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 608 aa, Artificial Sequence
  Exemplary sequence of a CD44v6-CAR28z, with spacer LNGFR wild-type short (NWS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 646 aa, Artificial Sequence
  Exemplary sequence of a CD44v6-CAR28z, with spacer LNGFR mutated long (NML)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 586 aa, Artificial Sequence
  Exemplary sequence of a CD44v6-CAR28z, with spacer LNGFR mutated short (NMS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 27 aa, Artificial Sequence
  transmembrane domain of CD28
FWVLVVVGGVLACYSLLVTVAFIIFWV

SEQ ID NO: 30 - protein, 41 aa, Artificial Sequence
  intracellular co-stimulatory domain of CD28
RSKRSRLLHSDYMNMTPRRPGPTRKHYQPYAPPRDFAAYRS

SEQ ID NO: 31 - protein, 251 aa, Artificial Sequence
  CD44v6-specific single chain fragment (CD44v6-4GS2)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSS

SEQ ID NO: 32 - protein, 658 aa, Artificial Sequence
  Sequence of CD44v6-4GS2-CAR28z, with spacer LNGFR wild-type long (NWL)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 598 aa, Artificial Sequence
  Sequence of CD44v6-4GS2-CAR28z, with spacer LNGFR wild-type short (NWS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 636 aa, Artificial Sequence
  Sequence of CD44v6-4GS2-CAR28z, with spacer LNGFR mutated long (NML)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEIPGRWITRSTPPEGSDSTAPSTQEPEAPPEQDLIASTVAGVVTTVMGSSQPVVTRGTTDNPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 576 aa, Artificial Sequence
  Sequence of CD44v6-4GS2-CAR28z, with spacer LNGFR mutated short (NMS)
MEAPAQLLFLLLLWLPDTTGEIVLTQSPATLSLSPGERATLSCSASSSINYIYWLQQKPGQAPRILIYLTSNLASGVPARFSGSGSGTDFTLTISSLEPEDFAVYYCLQWSSNPLTFGGGTKVEIKRGGGGSGGGGSEVQLVESGGGLVKPGGSLRLSCAASGFTFSSYDMSWVRQAPGKGLEWVSTISSGGSYTYYLDSIKGRFTISRDNAKNSLYLQMNSLRAEDTAVYYCARQGLDYWGRGTLVTVSSGDPKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEAARAADAECEEPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - protein, 10 aa, Artificial Sequence
  peptide linker
GGGGSGGGGS

SEQ ID NO: 37 - DNA, 8331 bp, Artificial Sequence
  Polynucleotide sequence of CD44v6-4GS2-CAR28z, with spacer LNGFR wild-type long (NWL)
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggaagcccctgcccagctgctgttcctgctgctgctgtggctgcccgacaccaccggcgagatcgtgctgacacagagccccgccaccctgtctctgagccctggcgagagagccaccctgagctgtagcgccagcagcagcatcaactacatctactggctgcagcagaagcccggccaggcccccagaatcctgatctacctgaccagcaacctggccagcggcgtgcccgccagattttctggcagcggcagcggcaccgacttcaccctgaccatcagcagcctggaacccgaggacttcgccgtgtactactgcctgcagtggtccagcaaccccctgaccttcggcggaggcaccaaggtggaaatcaagcggggtggtggtggttctggtggtggtggttctgaggtgcagctggtggaaagcggcggaggcctggtcaagcctggcggcagcctgagactgagctgtgccgccagcggcttcaccttcagcagctacgacatgagctgggtccgacaggctccaggcaagggactggaatgggtgtccaccatcagcagcggcggcagctacacctactacctggacagcatcaagggccggttcaccatcagccgggacaacgccaagaacagcctgtacctgcagatgaacagcctgcgggccgaggacaccgccgtctactactgtgcccggcagggcctcgactactggggcagaggcaccctggtcaccgtgtccagcggggatcccaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 38 - DNA, 8151 bp, Artificial Sequence
  Polynucleotide sequence of CD44v6-4GS2-CAR28z, with spacer LNGFR wild-type short (NWS)
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggaagcccctgcccagctgctgttcctgctgctgctgtggctgcccgacaccaccggcgagatcgtgctgacacagagccccgccaccctgtctctgagccctggcgagagagccaccctgagctgtagcgccagcagcagcatcaactacatctactggctgcagcagaagcccggccaggcccccagaatcctgatctacctgaccagcaacctggccagcggcgtgcccgccagattttctggcagcggcagcggcaccgacttcaccctgaccatcagcagcctggaacccgaggacttcgccgtgtactactgcctgcagtggtccagcaaccccctgaccttcggcggaggcaccaaggtggaaatcaagcggggtggtggtggttctggtggtggtggttctgaggtgcagctggtggaaagcggcggaggcctggtcaagcctggcggcagcctgagactgagctgtgccgccagcggcttcaccttcagcagctacgacatgagctgggtccgacaggctccaggcaagggactggaatgggtgtccaccatcagcagcggcggcagctacacctactacctggacagcatcaagggccggttcaccatcagccgggacaacgccaagaacagcctgtacctgcagatgaacagcctgcgggccgaggacaccgccgtctactactgtgcccggcagggcctcgactactggggcagaggcaccctggtcaccgtgtccagcggggatcccaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccaaccacgtggacccctgcctgccctgcactgtgtgcgaggacaccgagcggcagctgcgcgagtgcacaagatgggccgacgccgagtgcgaggaacccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 39 - DNA, 8265 bp, Artificial Sequence
  Polynucleotide sequence of CD44v6-4GS2-CAR28z, with spacer LNGFR mutated long (NML)
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggaagcccctgcccagctgctgttcctgctgctgctgtggctgcccgacaccaccggcgagatcgtgctgacacagagccccgccaccctgtctctgagccctggcgagagagccaccctgagctgtagcgccagcagcagcatcaactacatctactggctgcagcagaagcccggccaggcccccagaatcctgatctacctgaccagcaacctggccagcggcgtgcccgccagattttctggcagcggcagcggcaccgacttcaccctgaccatcagcagcctggaacccgaggacttcgccgtgtactactgcctgcagtggtccagcaaccccctgaccttcggcggaggcaccaaggtggaaatcaagcggggtggtggtggttctggtggtggtggttctgaggtgcagctggtggaaagcggcggaggcctggtcaagcctggcggcagcctgagactgagctgtgccgccagcggcttcaccttcagcagctacgacatgagctgggtccgacaggctccaggcaagggactggaatgggtgtccaccatcagcagcggcggcagctacacctactacctggacagcatcaagggccggttcaccatcagccgggacaacgccaagaacagcctgtacctgcagatgaacagcctgcgggccgaggacaccgccgtctactactgtgcccggcagggcctcgactactggggcagaggcaccctggtcaccgtgtccagcggggatcccaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaagacaagcagaataccgtgtgtgaagagtgccccgacggcacctacagcgacgaagccgccagagccgccgacgccgagtgcgaagagatccccggcagatggatcaccagaagcaccccccctgagggcagcgacagcaccgcccctagcacccaggaacctgaggcccctcccgagcaggacctgatcgcctctacagtggccggcgtggtgacaaccgtgatgggcagctctcagcccgtggtgacacggggcaccaccgacaatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 40 - DNA, 8085 bp, Artificial Sequence
  Polynucleotide sequence of CD44v6-4GS2-CAR28z, with spacer LNGFR mutated short (NMS)
aagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatggaagcccctgcccagctgctgttcctgctgctgctgtggctgcccgacaccaccggcgagatcgtgctgacacagagccccgccaccctgtctctgagccctggcgagagagccaccctgagctgtagcgccagcagcagcatcaactacatctactggctgcagcagaagcccggccaggcccccagaatcctgatctacctgaccagcaacctggccagcggcgtgcccgccagattttctggcagcggcagcggcaccgacttcaccctgaccatcagcagcctggaacccgaggacttcgccgtgtactactgcctgcagtggtccagcaaccccctgaccttcggcggaggcaccaaggtggaaatcaagcggggtggtggtggttctggtggtggtggttctgaggtgcagctggtggaaagcggcggaggcctggtcaagcctggcggcagcctgagactgagctgtgccgccagcggcttcaccttcagcagctacgacatgagctgggtccgacaggctccaggcaagggactggaatgggtgtccaccatcagcagcggcggcagctacacctactacctggacagcatcaagggccggttcaccatcagccgggacaacgccaagaacagcctgtacctgcagatgaacagcctgcgggccgaggacaccgccgtctactactgtgcccggcagggcctcgactactggggcagaggcaccctggtcaccgtgtccagcggggatcccaaagaggcctgccccaccggcctgtacacccacagcggagagtgctgcaaggcctgcaacctgggagagggcgtggcccagccttgcggcgccaatcagaccgtgtgcgagccctgcctggacagcgtgaccttcagcgacgtggtgtccgccaccgagccctgcaagccttgcaccgagtgtgtgggcctgcagagcatgagcgccccctgcgtggaagccgacgacgccgtgtgtagatgcgcctacggctactaccaggacgagacaaccggcagatgcgaggcctgtagagtgtgcgaggccggcagcggcctggtgttcagttgtcaggacaagcagaacaccgtgtgtgaagagtgccccgacggcacctacagcgacgaggccgcccgggccgccgacgccgagtgcgaggaacccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgcctcctcgctaagcatgcaacctcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgcc

SEQ ID NO: 41 - protein, 164 aa, Artificial Sequence
  Fusion protein conjugate
MCNGRCGVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 42 - DNA, 495 bp, Artificial Sequence
  polynucleotide sequence encoding a NGR-TNF conjugate
atgtgcaacggccgttgcggcgtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 43 - protein, 157 aa, Artificial Sequence [sequence omitted]

SEQ ID NO: 44 - protein, 5 aa, Artificial Sequence
  peptide comprising an NGR motif
  MISC_FEATURE (1)..(1): Xaa may be Leu, Val, Ala, Cys, Gly, Tyr, Pro, His, Lys, Gln or Ile
  MISC_FEATURE (5)..(5): Xaa may be Cys, Gly, His, Leu, Glu, Thr, Gln, Arg, Ser or Pro
XNGRX

SEQ ID NO: 45 - protein, 13 aa, Artificial Sequence
  peptide comprising an NGR motif
CNGRCVSGCAGRC

SEQ ID NO: 46 - protein, 6 aa, Artificial Sequence
  peptide comprising an NGR motif
NGRAHA

SEQ ID NO: 47 - protein, 5 aa, Artificial Sequence
  peptide comprising an NGR motif
GNGRG

SEQ ID NO: 48 - protein, 9 aa, Artificial Sequence
  peptide comprising an NGR motif
  MISC_FEATURE: may be cyclic peptide
CVLNGRMEC

SEQ ID NO: 49 - protein, 5 aa, Artificial Sequence
  peptide comprising an NGR motif
  MISC_FEATURE: may be cyclic peptide
CNGRC

SEQ ID NO: 50 - protein, 6 aa, Artificial Sequence
  peptide comprising an NGR motif
  MISC_FEATURE: may be cyclic peptide
GCNGRC

SEQ ID NO: 51 - protein, 6 aa, Artificial Sequence
  peptide comprising an NGR motif
  MISC_FEATURE: may be cyclic peptide
CNGRCG

SEQ ID NO: 52 - protein, 5 aa, Artificial Sequence
  peptide comprising an NGR motif
LNGRE

SEQ ID NO: 53 - protein, 5 aa, Artificial Sequence
  peptide comprising an NGR motif
YNGRT

SEQ ID NO: 54 - protein, 16 aa, Artificial Sequence
  peptide comprising an NGR motif
LQCICTGNGRGEWKCE

SEQ ID NO: 55 - protein, 16 aa, Artificial Sequence
  peptide comprising an NGR motif
LQCISTGNGRGEWKCE

SEQ ID NO: 56 - protein, 13 aa, Artificial Sequence
  peptide comprising an NGR motif
CICTGNGRGEWKC

SEQ ID NO: 57 - protein, 13 aa, Artificial Sequence
  peptide comprising an NGR motif
CISTGNGRGEWKC

SEQ ID NO: 58 - protein, 16 aa, Artificial Sequence
  peptide comprising an NGR motif
MRCTCVGNGRGEWTCY

SEQ ID NO: 59 - protein, 16 aa, Artificial Sequence
  peptide comprising an NGR motif
MRCTSVGNGRGEWTCY

SEQ ID NO: 60 - protein, 13 aa, Artificial Sequence
  peptide comprising an NGR motif
CTCVGNGRGEWTC

SEQ ID NO: 61 - protein, 13 aa, Artificial Sequence
  peptide comprising an NGR motif
CTSVGNGRGEWTC

SEQ ID NO: 62 - protein, 13 aa, Artificial Sequence
  targeting peptide containing the degradation product of the NGR motif
  MISC_FEATURE: may be cyclic peptide
  MOD_RES (2)..(2): isoD
CDGRCVSGCAGRC

SEQ ID NO: 63 - protein, 6 aa, Artificial Sequence
  targeting peptide containing the degradation product of the NGR motif
  MISC_FEATURE: may be cyclic peptide
  MOD_RES (1)..(1): isoD
DGRAHA

SEQ ID NO: 64 - protein, 5 aa, Artificial Sequence
  targeting peptide containing the degradation product of the NGR motif
  MISC_FEATURE: may be cyclic peptide
  MOD_RES (2)..(2): isoD
GDGRG

SEQ ID NO: 65 - protein, 9 aa, Artificial Sequence
  targeting peptide containing the degradation product of the NGR motif
  MISC_FEATURE: may be cyclic peptide
  MOD_RES (4)..(4): isoD
CVLDGRMEC

SEQ ID NO: 66 - protein, 5 aa, Artificial Sequence
  targeting peptide containing the degradation product of the NGR motif
  MISC_FEATURE: may be cyclic peptide
  MOD_RES (2)..(2): isoD
CDGRC

SEQ ID NO: 67 - protein, 6 aa, Artificial Sequence
  targeting peptide containing the degradation product of the NGR motif
  MISC_FEATURE: may be cyclic peptide
  MOD_RES (2)..(2): isoD
CDGRCG

SEQ ID NO: 68 - protein, 5 aa, Artificial Sequence
  targeting peptide containing the degradation product of the NGR motif
  MISC_FEATURE: may be cyclic peptide
  MOD_RES (2)..(2): isoD
LDGRE

SEQ ID NO: 69 - protein, 5 aa, Artificial Sequence
  targeting peptide containing the degradation product of the NGR motif
  MISC_FEATURE: may be cyclic peptide
  MOD_RES (2)..(2): isoD
YDGRT

SEQ ID NO: 70 - protein, 16 aa, Artificial Sequence
  targeting peptide containing the degradation product of the NGR motif
  MISC_FEATURE: may be cyclic peptide
  MOD_RES (8)..(8): isoD
LQCICTGDGRGEWKCE

SEQ ID NO: 71 - protein, 16 aa, Artificial Sequence
  targeting peptide containing the degradation product of the NGR motif
  MISC_FEATURE: may be cyclic peptide
  MOD_RES (8)..(8): isoD
LQCISTGDGRGEWKCE

SEQ ID NO: 72 - protein, 13 aa, Artificial Sequence
  targeting peptide containing the degradation product of the NGR motif
  MISC_FEATURE: may be cyclic peptide
  MOD_RES (6)..(6): isoD
CICTGDGRGEWKC

SEQ ID NO: 73 - protein, 13 aa, Artificial Sequence
  targeting peptide containing the degradation product of the NGR motif
  MISC_FEATURE: may be cyclic peptide
  MOD_RES (6)..(6): isoD
CISTGDGRGEWKC

SEQ ID NO: 74 - protein, 16 aa, Artificial Sequence
  targeting peptide containing the degradation product of the NGR motif
  MISC_FEATURE: may be cyclic peptide
  MOD_RES (8)..(8): isoD
MRCTCVGDGRGEWTCY

SEQ ID NO: 75 - protein, 16 aa, Artificial Sequence
  targeting peptide containing the degradation product of the NGR motif
  MISC_FEATURE: may be cyclic peptide
  MOD_RES (8)..(8): isoD
MRCTSVGDGRGEWTCY

SEQ ID NO: 76 - protein, 13 aa, Artificial Sequence
  targeting peptide containing the degradation product of the NGR motif
  MISC_FEATURE: may be cyclic peptide
  MOD_RES (6)..(6): isoD
CTCVGDGRGEWTC

SEQ ID NO: 77 - protein, 13 aa, Artificial Sequence
  targeting peptide containing the degradation product of the NGR motif
  MISC_FEATURE: may be cyclic peptide
  MOD_RES (6)..(6): isoD
CTSVGDGRGEWTC

(19) United States
(12) Patent Application Publication
(10) Pub. No.: US 2017/0252435 A1
(43) Pub. Date: Sep. 7, 2017

(54) Combination

(71) Applicant: MolMed SpA, Milan (IT)

(72) Inventors: Claudio Bordignon, Macugnaga (VB) (IT); Catia Traversari, Sesto San Giovanni (MI) (IT); Gian Paolo Rizzardi, Milan (IT)

(21) Appl. No.: 15/060,071

(22) Filed: Mar. 3, 2016

(51) Int. Cl. A61K 39/395; A61K 38/10; A61K 38/08; A61K 38/19; A61K 35/17

(52) U.S. Cl. CPC A61K 39/39558; A61K 38/191; A61K 35/17; A61K 38/08; A61K 38/10; A61K 2035/124

(57) ABSTRACT

A pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), wherein the CAR comprises an antigen-specific targeting domain which targets a tumour antigen.

### FIELD OF THE INVENTION

The present invention relates to products and methods for use in the treatment of cancer. In particular, the invention relates to combination therapies using tumour-targeted TNF and T cells genetically engineered to express chimeric antigen receptors (CARs).

### BACKGROUND TO THE INVENTION

The role of the immune system in the control of neoplastic growth has long been known and can be harnessed using adoptive immunotherapy strategies based on the transfer of antigen-specific T cells (Dotti, G. et al. (2014) Immunol. Rev. 257: 107-126). A major limitation of this approach is the small number of tumour-specific effectors that can be isolated from tumour histologies. As a result, use of naturally-occurring cells is severely restricted.

To overcome this limitation, new immunotherapies are being developed based on the use of genetically modified cells. For example, T cells may be genetically engineered ex vivo (e.g. using viral vectors) to express antigen receptors, such as chimeric antigen receptors (CARs).

CARs, which may comprise an engineered extracellular recognition domain and one or more intracellular signalling domains, are capable of redirecting the specificity of a T cell to an antigen expressed by cancer cells (Dotti, G. et al. (2014) Immunol. Rev. 257: 107-126; Jensen, M. C. et al. (2014) Immunol. Rev. 257: 127-144; and Gill, S. et al. (2015) Immunol. Rev. 263: 68-89). As a result, T cells which express suitable CARs are capable of killing tumour cells via the target antigen.

Therapeutic strategies using T cells expressing CARs directed to tumour antigens have been clinically tested for safety and efficacy. However, due to a lack of CARs able to recognise antigens expressed by solid tumours, and especially due to the difficulty in genetically modified T cells infiltrating tumour tissues, the majority of clinical studies conducted to date have used CARs specific for the CD19 antigen. This has limited their use to patients with haematological malignancies of lymphocyte line B.

Accordingly, their remains a significant need for improved therapeutic strategies for the treatment of cancer, in particular those which enable the treatment of solid tumours.

### SUMMARY OF THE INVENTION

The present invention relates to the development of combination therapy strategies to improve the effectiveness of cells, in particular lymphocytes genetically engineered to express chimeric antigen receptors (CARs). As used herein, the term “lymphocytes” includes T cells and natural killer cells, or a combination of both cell types. Specifically, the combination strategies of the invention include the use of tumour-targeted TNF, for example TNF conjugated to a peptide comprising an NGR moiety. The products and methods of the invention are particularly suitable for the treatment of solid tumours.

The tumour-targeted TNF may be, for example, administered simultaneously, sequentially or separately with cells which express a CAR. Different therapeutic windows (i.e. time periods between targeted TNF and T cell administration) associated with different therapeutic effects may be exploited.

While not wishing to be bound by theory, TNF conjugated to a peptide comprising an NGR moiety is understood to bind to tumour blood vessels and is associated with a transient improvement of vessel permeability (WO 2003/093478). In order to exploit this transient effect, cells (e.g. lymphocytes) expressing a CAR may be administered with or shortly after administration of the targeted TNF to enable more facile access of the lymphocytes to the tumour cells. For example, administration of the combination therapy components simultaneously or separated by a short period may avoid activation of potential counter regulatory mechanisms that may be associated with the administration of the TNF.

Alternatively or additionally, and again not wishing to be bound by theory, administration of TNF has also been associated with normalisation of tumour vessels (Porcellini, S. et al. (2015) Oncoimmunology 4: e1041700). This effect does not appear to be transient, but it can be observed for a number of days after administration of the TNF. In order to exploit this therapeutic window, cells (e.g. lymphocytes) expressing a CAR may be administered after a relatively long period following administration of the targeted TNF. This therapeutic window allows consolidated normalisation of tumour vessels, which promotes the best physiological conditions to obtain an effective immune response activated through CAR signalling.

Accordingly, in one aspect the invention provides a pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), wherein the CAR comprises an antigen-specific targeting domain which targets a tumour antigen.

In one embodiment, the pharmaceutical product is in the form of a pharmaceutical composition. Preferably, the pharmaceutical composition comprises a pharmaceutically acceptable carrier, diluent or excipient.

In another aspect, the invention provides the pharmaceutical product of the invention for use in therapy.

In another aspect, the invention provides a pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), as a combined preparation for simultaneous, sequential or separate use in therapy, wherein the CAR comprises an antigen-specific targeting domain which targets a tumour antigen.

In another aspect, the invention provides a method of treating cancer comprising administering (a) a conjugation product of TNF and a tumour- or tumour vasculature-targeting peptide comprising an NGR, DGR, isoDGR or RGD motif; and (b) a cell comprising a chimeric antigen receptor (CAR), to a subject simultaneously, sequentially or separately.

The time between the administration of the conjugation product and the cells comprising the CAR may be, for example, adjusted to utilise different therapeutic windows, which may be associated with different therapeutic effects.

In one embodiment, the cell comprising a CAR is administered to the subject about 1-10, 1-9, 1-8, 1-7, 1-6, 1-5, 1-4, 1-3 or 1-2 hours, preferably about 1-4 hours, after administration of the conjugation product of TNF and a tumour- or tumour vasculature-targeting peptide.

In another embodiment, the cell comprising a CAR is administered to the subject about 10, 9, 8, 7, 6, 5, 4, 3, 2 or 1 hours, preferably about 4, 3, 2 or 1 hours, after administration of the conjugation product of TNF and a tumour- or tumour vasculature-targeting peptide.

In another embodiment, the cell comprising a CAR is administered to the subject more than about 12, 24, 48, 72 or 96 hours, or more than about 1 week after administration of the conjugation product of TNF and a tumour- or tumour vasculature-targeting peptide.

In another embodiment, the cell comprising a CAR is administered to the subject about 12-168, 12-96, 12-72, 12-48 or 12-24 hours after administration of the conjugation product of TNF and a tumour- or tumour vasculature-targeting peptide.

In another embodiment, the cell comprising a CAR is administered to the subject about 12, 24, 48, 72 or 96 hours, or about 1 week after administration of the conjugation product of TNF and a tumour- or tumour vasculature-targeting peptide.

In one embodiment, the conjugation product of TNF and a tumour- or tumour vasculature-targeting peptide comprising an NGR, DGR, isoDGR or RGD motif is administered to the subject in 1, 2, 3, 4 or 5 doses, preferably 3 doses, over about a 1-3 week, preferably 1 week, period.

Preferably, the cell comprising a CAR is only administered to the subject after the final dose of the conjugation product of TNF and a tumour- or tumour vasculature-targeting peptide comprising an NGR, DGR, isoDGR or RGD motif.

In one embodiment, the conjugation product of TNF and a tumour- or tumour vasculature-targeting peptide comprising an NGR, DGR, isoDGR or RGD motif is administered at a dosage of 0.5-500 ng/kg, 1-100 ng/kg, 1-50 ng/kg or 5-15 ng/kg. In a preferred embodiment, the conjugation product of TNF and a peptide comprising an NGR, DGR, isoDGR or RGD motif is administered at a dose of 0.8 μg/m².

In one embodiment, the tumour-targeting peptide is a binding partner of a tumour receptor, marker or other extracellular component.

In one embodiment, the tumour vasculature-targeting peptide is a binding partner of a tumour vasculature receptor, marker or other extracellular component.

In a preferred embodiment, the tumour- or tumour vasculature-targeting peptide is a peptide comprising an NGR motif.

The peptide (e.g. comprising the NGR motif) may comprise, for example, up to 350, up to 100, up to 50, up to 25 or up to 15 amino acids.

In one embodiment, the peptide comprising an NGR motif comprises the sequence XNGRX′, wherein X is selected form the group consisting of L, V, A, C, G, Y, P, H, K, Q and I, and X′ is selected from the group consisting of C, G, H, L, E, T, Q, R, S and P.

In another embodiment, the peptide comprising an NGR motif comprises a sequence selected from the group consisting of CNGRCVSGCAGRC, NGRAHA, GNGRG, CVLNGRMEC, CNGRC, GCNGRC, CNGRCG, LNGRE, YNGRT, LQCICTGNGRGEWKCE, LQCISTGNGRGEWKCE, CICTGNGRGEWKC, CISTGNGRGEWKC, MRCTCVGNGRGEWTCY, MRCTSVGNGRGEWTCY, CTCVGNGRGEWTC and CTSVGNGRGEWTC.

In one embodiment, the peptide comprising the NGR motif consists of a sequence selected from the group consisting of CNGRCVSGCAGRC, NGRAHA, GNGRG, CVLNGRMEC, CNGRC, CNGRCG, GCNGRC, LNGRE, YNGRT, LQCICTGNGRGEWKCE, LQCISTGNGRGEWKCE, CICTGNGRGEWKC, CISTGNGRGEWKC, MRCTCVGNGRGEWTCY, MRCTSVGNGRGEWTCY CTCVGNGRGEWTC and CTSVGNGRGEWTC.

In another embodiment, the peptide comprising an NGR motif comprises a sequence selected from the group consisting of cycloCVLNGRMEC, linear CNGRC, cyclic CNGRC, linear GCNGRC, linear CNGRCG, cyclic GCNGRC and cyclic CNGRCG.

In another embodiment, the peptide comprising an NGR motif consists of a sequence selected from the group consisting of cycloCVLNGRMEC, linear CNGRC, cyclic CNGRC, linear GCNGRC, linear CNGRCG, cyclic GCNGRC and cyclic CNGRCG.

In a preferred embodiment, the peptide comprising an NGR motif comprises the sequence CNGRCG or GCNGRC. The CNGRCG or GCNGRC may be linear or cyclic, preferably cyclic. Preferably, the peptide comprising an NGR motif comprises the sequence CNGRCG.

Preferably, the conjugate is in the form of a fusion protein. Preferably, the conjugate is a fusion of CNGRCG with the N-terminus of TNF. CNGRCG provides high specificity targeting to tumour blood vessels (WO 2001/061017).

Preferably, the conjugate has the following amino acid sequence:

MCNGRCGVRSSSRTPSDKPVAHVVANPQAEGQLQWLNRRANALLANGVEL
RDNQLVVPSEGLYLIYSQVLFKGQGCPSTHVLLTHTISRIAVSYQTKVNL
LSAIKSPCQRETPEGAEAKPWYEPIYLGGVFQLEKGDRLSAEINRPDYLD
FAESGQVYFGIIAL (SEQ ID NO: 41; the underlined
amino acids correspond to the targeting peptide)

An example polynucleotide sequence encoding a NGR-TNF conjugate is:

ATGtgcaacggccgttgcggcgtcagatcatcttctcgaaccccgagtga
caagcctgtagcccatgttgtagcaaaccctcaagctgaggggcagctcc
agtggctgaaccgccgggccaatgccctcctggccaatggcgtggagctg
agagataaccagctggtggtgccatcagagggcctgtacctcatctactc
ccaggtcctcttcaagggccaaggctgcccctccacccatgtgctcctca
cccacaccatcagccgcatcgccgtctcctaccagaccaaggtcaacctc
ctctctgccatcaagagcccctgccagagggagaccccagagggggctga
ggccaagccctggtatgagcccatctatctgggaggggtcttccagctgg
agaagggtgaccgactcagcgctgagatcaatcggcccgactatctcgac
tttgccgagtctgggcaggtctactttgggatcattgccctgTGA
(SEQ ID NO: 42; the underlined nucleotides
correspond to the nucleotides encoding the
targeting peptide)

In one embodiment, the cell is a lymphocyte, such as a T cell or a natural killer cell, preferably a T cell.

In another embodiment, the cell is a haematopoietic stem cell and/or a cell capable of giving rise to therapeutically relevant progeny, preferably a haematopoietic stem cell.

In one embodiment, the TNF is TNFα or INFβ, preferably TNFα. Preferably, the TNF is human or murine TNF, more preferably human TNF.

In one embodiment, the TNF is derivatised with polyethylene glycol or an acyl residue.

In one embodiment, the conjugation product of TNF and a tumour- or tumour vasculature-targeting peptide comprising an NGR, DGR, or RGD motif of the invention is in the form of nucleic acid. Thus, according to another aspect the invention provides a pharmaceutical product comprising (a) a polynucleotide encoding a conjugation product of TNF and a tumour- or tumour vasculature-targeting peptide comprising an NGR, DGR, or RGD motif; and (b) a cell comprising a chimeric antigen receptor (CAR), wherein the CAR comprises an antigen-specific targeting domain which targets a tumour antigen.

The nucleic acid may be in the form of a vector, preferably a viral vector. Preferably, the viral vector is in the form of a viral vector particle.

In one embodiment, the CAR comprises (a) an antigen-specific targeting domain; (b) an extracellular spacer domain; (c) a transmembrane domain; (d) optionally, at least one co-stimulatory domain; and (e) an intracellular signalling domain.

In one embodiment, the CAR comprises an antigen-specific targeting domain comprising an antibody or fragment thereof.

In another embodiment, the CAR comprises an antigen-specific targeting domain which is a single chain variable fragment.

In one embodiment, the CAR comprises an antigen-specific targeting domain which targets a tumour antigen, wherein the tumour antigen is selected from the group consisting of CD44, CD19, CD20, CD22, CD23, CD123, CS-1, ROR1, mesothelin, c-Met, PSMA, Her2, GD-2, CEA, MAGE A3 TCR and combinations thereof. As used herein, the term “tumour antigen” means an antigen expressed by a tumour.

In a preferred embodiment, the tumour antigen is isoform 6 of CD44 (CD44v6).

In one embodiment, the CAR comprises a transmembrane domain comprising any one or more of a transmembrane domain of a zeta chain of a T cell receptor complex, CD28, CD8a and combinations thereof. Preferably, the transmembrane domain comprises a transmembrane domain of CD28.

In one embodiment, the CAR comprises a co-stimulatory domain comprising a co-stimulating domain from any one or more of CD28, CD137 (4-1 BB), CD134 (OX40), DapIO, CD27, CD2, CD5, ICAM-1, LFA-1, Lck, TNFR-I, TNFR-II, Fas, CD30, CD40 and combinations thereof. Preferably, the co-stimulatory domain comprises a CD28 endo-co-stimulating domain.

In one embodiment, the CAR comprises an intracellular signalling domain comprising an intracellular signalling domain of any one or more of a human CD3 zeta chain, FcyRIII, FcsRI, a cytoplasmic tail of a Fc receptor, an immunoreceptor tyrosine-based activation motif (ITAM) bearing cytoplasmic receptors and combinations thereof. Preferably, the intracellular signalling domain comprises an intracellular signalling domain of human CD3 zeta chain.

In a preferred embodiment, the CAR comprises an antigen-specific targeting domain which targets CD44v6; a transmembrane domain comprising a transmembrane domain of CD28; an intracellular signalling domain comprising an intracellular signalling domain of human CD3 zeta chain; and a co-stimulatory domain comprising a CD28 endo-co-stimulating domain.

In a preferred embodiment, the CAR comprises an extracellular spacer comprising at least part of the extracellular domain of human low affinity nerve growth factor receptor (LNGFR) or a derivative thereof. In one embodiment, the CAR comprises an extracellular spacer comprising the extracellular domain of human low affinity nerve growth factor receptor (LNGFR).

The CAR may comprise at least a fragment of the extracellular domain of the human low affinity nerve growth factor receptor (LNGFR) or a derivative thereof.

Preferably, the at least part of the LNGFR facilitates immunoselection and/or identification of cells transduced with the CAR.

In one embodiment, the extracellular spacer lacks the intracellular domain of LNGFR.

In one embodiment, the extracellular spacer comprises the first three TNFR-Cys domains of LNGFR, or fragments or derivatives thereof.

In one embodiment, the extracellular spacer comprises all four TNFR-Cys domains of LNGFR, or fragments or derivatives thereof.

In one embodiment, the extracellular spacer comprises the fourth TNFR-Cys domain (TNFR-Cys 4), but wherein the following amino acid sequence is removed from said domain: NHVDPCLPCTVCEDTERQLRECTRW (SEQ ID NO: 13). Preferably, the NHVDPCLPCTVCEDTERQLRECTRW sequence is replaced with the following amino acid sequence: ARA.

In one embodiment, the extracellular spacer comprises the serine/threonine-rich stalk of LNGFR.

In another embodiment, the extracellular spacer lacks the serine/threonine-rich stalk of LNGFR.

In one embodiment, the extracellular spacer comprises the entire extracellular domain of LNGFR.

In one embodiment, the extracellular spacer comprises the extracellular domain of LNGFR with the exception of the serine/threonine-rich stalk of said domain.

In one embodiment, the extracellular spacer comprises a sequence selected from the group consisting of SEQ ID NO: 1, SEQ ID NO: 3, SEQ ID NO: 5 or SEQ ID NO: 7, or a sequence at least 80%, 85%, 90%, 95%, 96%, 97%, 98% or 99% identical thereto.

In one embodiment, the extracellular spacer consists of a sequence selected from the group consisting of SEQ ID NO: 1, SEQ ID NO: 3, SEQ ID NO: 5 or SEQ ID NO: 7, or a sequence at least 80%, 85%, 90%, 95%, 96%, 97%, 98% or 99% identical thereto.

SEQ ID NO: 1, SEQ ID NO: 3 and SEQ ID NO: 5 are preferred spacer elements.

In another aspect, the invention provides the use of (a) a conjugation product of TNF and a tumour- or tumour vasculature-targeting peptide comprising an NGR, DGR, isoDGR or RGD motif; and (b) a cell comprising a chimeric antigen receptor (CAR) for the manufacture of a medicament for treating cancer, wherein the CAR comprises an antigen-specific targeting domain which targets a tumour antigen.

In one embodiment, the medicament is in the form of a pharmaceutical composition.

In another aspect, the invention provides the use of a conjugation product of TNF and a tumour- or tumour vasculature-targeting peptide comprising an NGR, DGR, isoDGR or RGD motif for the manufacture of a medicament for treating cancer, wherein the treatment comprises the simultaneous, sequential or separate administration to a subject of the conjugation product of TNF and a tumour- or tumour vasculature-targeting peptide comprising an NGR, DGR, isoDGR or RGD motif, and a cell comprising a chimeric antigen receptor (CAR), wherein the CAR comprises an antigen-specific targeting domain which targets a tumour antigen.

In another aspect, the invention provides the use of a cell comprising a chimeric antigen receptor (CAR) for the manufacture of a medicament for treating cancer, wherein the treatment comprises the simultaneous, sequential or separate administration to a subject of the cell comprising a CAR, and a conjugation product of TNF and a tumour- or tumour vasculature-targeting peptide comprising an NGR, DGR, isoDGR or RGD motif, wherein the CAR comprises an antigen-specific targeting domain which targets a tumour antigen.

In another aspect, the invention provides the use of a conjugation product of TNF and a tumour- or tumour vasculature-targeting peptide comprising an NGR, DGR, isoDGR or RGD motif for the manufacture of a medicament for treating cancer, wherein the medicament is for use in combination therapy with a cell comprising a chimeric antigen receptor (CAR), wherein the CAR comprises an antigen-specific targeting domain which targets a tumour antigen.

In another aspect, the invention provides the use of a cell comprising a chimeric antigen receptor (CAR) for the manufacture of a medicament for treating cancer, wherein the medicament is for use in combination therapy with a conjugation product of TNF and a tumour- or tumour vasculature-targeting peptide comprising an NGR, DGR, isoDGR or RGD motif, wherein the CAR comprises an antigen-specific targeting domain which targets a tumour antigen.

The product, tumour- or tumour-vasculature-targeting peptide, TNF, cell, CAR and mode of treatment may be as described herein.

### DESCRIPTION OF THE DRAWINGS

FIG. 1. Sequence of human LNGFR.

FIG. 2. Sequence of CD44v6CAR.28z. The SCFV, CH2CH3, CD28 and zeta chain sequences are shown.

FIG. 3. Exemplary sequence of a CD44v6CAR.28z, with spacer LNGFR wild-type long (NWL) (SEQ ID NO:21)

FIG. 4. Exemplary sequence of a CD44v6-CAR28z, with spacer LNGFR wild-type short (NWS) (SEQ ID NO:22)

FIG. 5. Exemplary sequence of a CD44v6-CAR28z, with spacer LNGFR mutated long (NML) (SEQ ID NO:23)

FIG. 6. Exemplary sequence of a CD44v6-CAR28z, with spacer LNGFR mutated short (NMS) (SEQ ID NO:24)

FIG. 7. Exemplary sequence of a CD44v6CAR.28z, with spacer LNGFR wild-type long (NWL) (SEQ ID NO:25)

FIG. 8. Exemplary sequence of a CD44v6-CAR28z, with spacer LNGFR wild-type short (NWS) (SEQ ID NO:26)

FIG. 9. Exemplary sequence of a CD44v6-CAR28z, with spacer LNGFR mutated long (NML) (SEQ ID NO:27)

FIG. 10. Exemplary sequence of a CD44v6-CAR28z, with spacer LNGFR mutated short (NMS) (SEQ ID NO:28)

FIG. 11. Sequence of CD44v6-4GS2-CAR28z, with spacer LNGFR wild-type long (NWL) (SEQ ID NO:32)

FIG. 12. Sequence of CD44v6-4GS2-CAR28z, with spacer LNGFR wild-type short (NWS) (SEQ ID NO:33)

FIG. 13. Sequence of CD44v6-4GS2-CAR28z, with spacer LNGFR mutated long (NML) (SEQ ID NO:34)

FIG. 14. Sequence of CD44v6-4GS2-CAR28z, with spacer LNGFR mutated short (NMS) (SEQ ID NO:35)

FIG. 15. Polynucleotide sequence of CD44v6-4GS2-CAR28z, with spacer LNGFR wild-type long (NWL) (SEQ ID NO:37)

FIG. 16. Polynucleotide sequence of CD44v6-4GS2-CAR28z, with spacer LNGFR wild-type short (NWS) (SEQ ID NO:38)

FIG. 17. Polynucleotide sequence of CD44v6-4GS2-CAR28z, with spacer LNGFR mutated long (NML) (SEQ ID NO:39)

FIG. 18. Polynucleotide sequence of CD44v6-4GS2-CAR28z, with spacer LNGFR mutated short (NMS) (SEQ ID NO:40)

### DETAILED DESCRIPTION OF THE INVENTION

Various preferred features and embodiments of the present invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J. O′D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### Pharmaceutical Product

The pharmaceutical product of the present invention may be, for example, a pharmaceutical composition comprising (a) a conjugation product of TNF and a tumour- or tumour vasculature-targeting peptide comprising an NGR, DGR, isoDGR or RGD motif (herein referred to as a TNF conjugate); and (b) a cell comprising a chimeric antigen receptor (CAR).

Alternatively, the pharmaceutical product may be, for example, a kit comprising a preparation of a cell comprising a chimeric antigen receptor (CAR), and a TNF conjugate, and, optionally, instructions for the simultaneous, sequential or separate administration of the preparations to a subject in need thereof.

Preferably, the combination of the TNF conjugate and the cell comprising a chimeric antigen receptor (CAR) has a synergistic effect, i.e. the combination is synergistic.

As used herein, the term “combination therapy” refers to therapy in which the TNF conjugate and the cell comprising a CAR, are administered, if not simultaneously, then sequentially within a timeframe that they both are available to act therapeutically within the same time-frame.

As mentioned above, one aspect of the invention relates to a pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), as a combined preparation for simultaneous, sequential or separate use in therapy.

The TNF conjugate and the cell comprising a CAR may be administered simultaneously, in combination, sequentially or separately (as part of a dosing regime).

As used herein, the term “simultaneously” means that the two agents are administered concurrently, whereas the term “in combination” means that they are administered, if not simultaneously, then “sequentially” within a time-frame that they are both available to act therapeutically within the same time-frame. Thus, administration “sequentially” may permit one agent to be administered within 5 minutes, 10 minutes or a matter of hours after the other provided the circulatory half-life of the first administered agent is such that they are both concurrently present in therapeutically effective amounts. The time delay between administration of the components will vary depending on the exact nature of the components, the interaction there-between, and their respective half-lives.

In contrast to “in combination” or “sequentially”, the term “separately” means that the gap between administering one agent and the other is significant. For example, the first administered agent may no longer be present in the bloodstream in a therapeutically effective amount when the second agent is administered.

### Tumour Necrosis Factor (TNF)

Tumour necrosis factor (TNF) acts as an inflammatory cytokine and has the effect of inducing alteration of the endothelial barrier function, reducing tumour interstitial pressure, and increasing chemotherapeutic drug penetration and tumour vessel damage.

The first suggestion that a tumour necrotising molecule existed was made when it was observed that cancer patients occasionally showed spontaneous regression of their tumours following bacterial infections. Subsequent studies in the 1960s indicated that host-associated (or endogenous) mediators, manufactured in response to bacterial products, were likely responsible for the observed effects. In 1975 it was shown that a bacterially-induced circulating factor had strong anti-tumour activity against tumours implanted in the skin in mice. This factor, designated TNF, was subsequently isolated, cloned, and found to be the prototype of a family of molecules that are involved with immune regulation and inflammation. The receptors for TNF and the other members of the TNF superfamily also constitute a superfamily of related proteins.

Human TNFα is a 233 amino acid residue, non-glycosylated polypeptide that exists as either a transmembrane or soluble protein. When expressed as a 26 kDa membrane-bound protein, TNFα consists of a 29 amino acid residue cytoplasmic domain, a 28 amino acid residue transmembrane segment, and a 176 amino acid residue extracellular region. The soluble protein is created by a proteolytic cleavage event via an 85 kDa TNFα converting enzyme (TACE), which generates a 17 kDa, 157 amino acid residue molecule that normally circulates as a homotrimer.

The sequence of human TNFα can be found at UniProtKB/Swiss-Prot: P01375.

The sequence of mouse TNFα can be found at UniProtKB/Swiss-Prot: P06804.

An example TNF amino acid sequence is:

(SEQ ID NO: 43)
VRSSSRTPSDKPVAHVVANPQAEGQLQWLNRRANALLANGVELRDNQLVV
PSEGLYLIYSQVLFKGQGCPSTHVLLTHTISRIAVSYQTKVNLLSAIKSP
CQRETPEGAEAKPWYEPIYLGGVFQLEKGDRLSAEINRPDYLDFAESGQV
YFGIIAL

TNFβ, otherwise known as lymphotoxin-α (LT-α) is a molecule whose cloning was contemporary with that of TNFα. Although TNFβ circulates as a 171 amino acid residue, 25 kDa glycosylated polypeptide, a larger form has been found that is 194 amino acid residues long. The human TNFβ cDNA codes for an open reading frame of 205 amino acid residues (202 in the mouse), and presumably some type of proteolytic processing occurs during secretion. As with TNFα, circulating TNFβ exists as a non-covalently linked trimer and is known to bind to the same receptors as TNFα.

The sequence of human TNFβ can be found at UniProtKB/Swiss-Prot: P01374.

The sequence of mouse TNFβ can be found at UniProtKB/Swiss-Prot: P01374

The maximum tolerated dose of bolus TNF in humans is 218-410 μg/m² (Fraker, D. L. et al. (1995) Biologic therapy of cancer: principles and practice, 329-345, L.B. Lippincott Company) about 10-fold lower than the effective dose in animals. Based on data from murine models it is believed that an at least 10-times higher dose is necessary to achieve anti-tumour effects in humans (Schraffordt Koops et al. (1998) Radiotherapy Oncology 48: 1-4).

It is known that TNF can decrease the barrier function of the endothelial lining vessels, thus increasing their permeability to macromolecules. Thus, the pharmaceutical product of the present invention may be used to increase the permeability of tumour vessels to other compounds, either for therapeutic or diagnostic purposes. For instance, the pharmaceutical product may be used to increase the tumour uptake of radiolabelled antibodies or hormones (tumour-imaging compounds) in radioimmunoscintigraphy or radioimmunotherapy of tumours. Alternatively, the uptake of chemotherapeutic drugs, immunotoxins, liposomes carrying drugs or genes, or other anticancer drugs could also be increased, so that their anti-tumour effects are enhanced.

TNF, as referred to herein, includes fragments, variants and derivatives of TNF, which retain the physiological activity of TNF. Preferably, the fragments, variants and derivatives, when conjugated to a targeting moiety, retain at least 25%, 50%, 75% or 95% of the anti-tumour activity of the corresponding wild-type TNF conjugate when used in a pharmaceutical product of the present invention. Preferably, the fragments, variants and derivatives of the full length TNF cytokine are capable of selectively binding to one of the TNF receptors (Loetscher, H. et al. (1993) J. Biol. Chem. 268:26350-7; Van Ostade, X. et al. (1993) Nature 361:266-9).

### Conjugates

The pharmaceutical product of the present invention comprises, in addition to a cell comprising a chimeric antigen receptor (CAR), a targeting moiety (i.e. the tumour- or tumour vasculature-targeting peptide comprising an NGR, DGR, isoDGR or RGD motif) linked to TNF.

The conjugate may be a molecule comprising at least one targeting moiety linked to TNF formed through genetic fusion or chemical coupling. Preferably, the targeting moiety is a polypeptide. By “linked” it is to be understood that the first and second sequences are associated such that the second sequence is able to be transported by the first sequence to a target cell. Thus, conjugates include fusion proteins in which the targeting moiety is linked to TNF via their polypeptide backbones through genetic expression of a DNA molecule encoding these proteins, directly synthesised proteins and coupled proteins in which pre-formed sequences are associated by a cross-linking agent. The term is also used herein to include associations, such as aggregates, of the cytokine with the targeting moiety.

The targeting moiety can be coupled directly to the TNF or indirectly through a spacer, which can be a single amino acid (e.g. G (glycine)), an amino acid sequence or an organic residue, such as 6-aminocapryl-N-hydroxysuccinimide.

The targeting moiety is preferably linked to the TNF N-terminus, thus minimising any interference in the binding of the TNF to its receptor. Alternatively, the peptide can be linked to amino acid residues, which are amido- or carboxylic-bond acceptors, which may be naturally occurring on the molecule or artificially inserted using genetic engineering techniques. The modified TNF is preferably prepared by use of a cDNA comprising a 5′-contiguous sequence encoding the peptide.

In a particularly preferred embodiment, targeted delivery of TNF can be achieved with a targeting moiety comprising a peptide containing the NGR motif, such as a modified ligand of aminopeptidase-N receptor (CD13). Such ligands are described in WO 1998/010795, which is herein incorporated by reference. Methods of identifying ligands of the CD13 receptor are disclosed in WO 1999/013329, which is herein incorporated by reference.

CD13 is a trans-membrane glycoprotein of 150 kDa highly conserved in various species. It is expressed on normal cells as well as in myeloid tumour lines, in the angiogenic endothelium and in some epithelia. CD13 receptor is usually identified as “NGR” receptor, in that its peptide ligands share the amino acidic “NGR” motif.

The pharmacokinetic properties of the conjugates used in the invention can be improved by preparing polyethylene glycol derivatives, which extend the plasmatic half-life of the cytokines themselves.

### CD13

It has been found that the therapeutic index of certain cytokines can be remarkably improved and their immunotherapeutic properties can be enhanced by coupling with a ligand of aminopeptidase-N receptor (CD13). CD13 is a transmembrane glycoprotein of 150 kDa that is highly conserved in various species. It is expressed on normal cells as well as in myeloid tumour lines, in the angiogenic endothelium and in some epithelia. The CD13 receptor is usually identified as “NGR” receptor, in that its peptide ligands share the amino acidic “NGR” motif.

In a particularly preferred embodiment, targeted delivery of TNF can be achieved with a targeting moiety comprising a peptide containing the NGR motif. Such ligands are described in WO 1998/010795, which is herein incorporated by reference. Methods of identifying ligands of the CD13 receptor are disclosed in WO 1999/013329, which is herein incorporated by reference.

The NGR motif comprises a turn involving the G and R residues. The structure-activity relationship of linear and cyclic peptides containing the NGR motif and their ability to target tumours is discussed in Colombo et al. (Colombo et al. (2002) J. Biol. Chem. 49: 47891-47897). The experiments carried out in animal models showed that both GNGRG and CNGRC can target TNF to tumours. Molecular dynamic simulation of cyclic CNGRC showed the presence of a bend geometry involving residues Gly³-Arg⁴, stabilised by the formation of a disulfide bridge. Molecular dynamic simulation of the same peptide without disulfide constraints showed that the most populated and thermodynamically favoured configuration is characterised by the presence of a β-turn involving residues Gly³-Arg⁴. These results suggest that the NGR motif has a strong propensity to form a β-turn in linear peptides.

### Integrins

An integrin molecule is composed of two noncovalently associated transmembrane glycoprotein subunits called a and R. Because the same integrin molecule in different cell types can have different ligand-binding specificities, it seems that additional cell-type-specific factors can interact with integrin modulate their binding activity. α and β subunits can combine in different ways to form integrin receptors. Natural ligands of integrin are adhesive proteins of the extracellular matrix proteins such as fibronectin, vitronectin, collagens, laminin.

Many integrins, particularly αvβ3 integrin, recognise the amino acid sequence RGD (arginine-glycine-aspartic acid). In a further embodiment, the targeting peptide is a peptide able to bind to the αvβ3 integrin, particularly a peptide containing the RDG motif.

Other ligands of αvβ3 integrin are peptides containing degradation products of the NGR motif. Details of these peptides are disclosed in WO 2006/067633, incorporated herein by reference. In a further embodiment, the targeting peptides are peptides containing the degradation product of the NGR motif, particularly peptides containing the isoDGR motif.

In a particularly preferred embodiment, the targeting peptides are selected from the group consisting of linear or cyclic CisoDGRCVSGCAGRC, isoDGRAHA, GisoDGRG, CVLisoDGRMEC, CisoDGRC, CisoDGRCG, LisoDGRE, YisoDGRT, LQCICTGisoDGRGEWKCE, LQCISTGisoDGRGEWKCE, CICTGisoDGRGEWKC, CISTGisoDGRGEWKC, MRCTCVGisoDGRGEWTCY, MRCTSVGisoDGRGEWTCY, CTCVGisoDGRGEWTC or CTSVGisoDGRGEWTC.

### Binding Partner (BP)

The targeting peptide of the invention generally takes the form of a binding partner (BP) to a surface molecule comprising or consisting of one or more binding domains.

### Ligand

The targeting peptide of the invention may take the form of a ligand. The ligands may be natural or synthetic. The term “ligand” also refers to a chemically-modified ligand. The one or more binding domains of the BP may consist of, for example, a natural ligand for a receptor, which natural ligand may be an adhesion molecule or a growth-factor receptor ligand (e.g. epidermal growth factor), or a fragment of a natural ligand which retains binding affinity for the receptor.

Synthetic ligands include the designer ligands. As used herein, the term “designer ligands” refers to agents which are likely to bind to the receptor based on their three dimensional shape compared to that of the receptor.

### Chimeric Antigen Receptors (CARs)

The term “chimeric antigen receptor” (CAR) as used herein refers to engineered receptors which can confer an antigen specificity onto cells (for example lymphocytes, such as natural killer cells or T cells, including naive T cells, central memory T cells, effector memory T cells or combinations thereof). CARs are also known as artificial T cell receptors, chimeric T cell receptors or chimeric immunoreceptors. Preferably, the CARs of the invention comprise an antigen-specific targeting region, an extracellular domain, a transmembrane domain, optionally one or more co-stimulatory domains and an intracellular signalling domain.

### Antigen-Specific Targeting Domain

The antigen-specific targeting domain provides the CAR with the ability to bind to the target antigen of interest. The antigen-specific targeting domain preferably targets an antigen of clinical interest against which it would be desirable to trigger an effector immune response that results in tumour killing.

The antigen-specific targeting domain may be any protein or peptide that possesses the ability to specifically recognise and bind to a biological molecule (e.g. a cell surface receptor or tumour protein, or a component thereof). The antigen-specific targeting domain includes any naturally-occurring, synthetic, semi-synthetic or recombinantly-produced binding partner for a biological molecule of interest.

Illustrative antigen-specific targeting domains include antibodies or antibody fragments or derivatives, extracellular domains of receptors, ligands for cell surface molecules/receptors, or receptor binding domains thereof, and tumour-binding proteins.

In a preferred embodiment, the antigen-specific targeting domain is, or is derived from, an antibody. An antibody-derived targeting domain can be a fragment of an antibody or a genetically engineered product of one or more fragments of the antibody, which fragment is involved in binding with the antigen. Examples include a variable region (Fv), a complementarity determining region (CDR), a Fab, a single chain antibody (scFv), a heavy chain variable region (VH), a light chain variable region (VL) and a camelid antibody (VHH).

In a preferred embodiment, the binding domain is a single chain antibody (scFv). The scFv may be a murine, human or humanised scFv.

The term “complementarity determining region” (CDR), with regard to an antibody or antigen-binding fragment thereof, refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs.

The term “heavy chain variable region” (VH) refers to the fragment of the heavy chain of an antibody that contains three CDRs interposed between flanking stretches known as framework regions, which are more highly conserved than the CDRs and form a scaffold to support the CDRs.

The term “light chain variable region” (VL) refers to the fragment of the light chain of an antibody that contains three CDRs interposed between framework regions.

The term “Fv” refers to the smallest fragment of an antibody to bear the complete antigen binding site. An Fv fragment consists of the variable region of a single light chain bound to the variable region of a single heavy chain.

The term “single-chain Fv antibody” (scFv) refers to an engineered antibody consisting of a light chain variable region and a heavy chain variable region connected to one another directly or via a peptide linker sequence.

Antibodies that specifically bind a tumour cell surface molecule can be prepared using methods well known in the art. Such methods include phage display, methods to generate human or humanised antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

Examples of antigens which may be targeted by the CAR of the invention include but are not limited to antigens expressed on cancer cells and antigens expressed on cells associated with various haematologic diseases, autoimmune diseases, inflammatory diseases and infectious diseases.

With respect to targeting domains that target cancer antigens, the selection of the targeting domain will depend on the type of cancer to be treated, and may target tumour antigens. A tumour sample from a subject may be characterised for the presence of certain biomarkers or cell surface markers. For example, breast cancer cells from a subject may be positive or negative for each of Her2Neu, estrogen receptor and/or the progesterone receptor. A tumour antigen or cell surface molecule is selected that is found on the individual subject's tumour cells. Preferably, the antigen-specific targeting domain targets a cell surface molecule that is found on tumour cells and is not substantially found on normal tissues or restricted in its expression to non-vital normal tissues.

Further antigens specific for cancer which may be targeted by the CAR of the invention include but are not limited to any one or more of carcinoembryonic antigen (CEA), prostate specific antigen, PSMA, Her2/neu, estrogen receptor, progesterone receptor, ephrinB2, ROR1, mesothelin, c-Met, GD-2, MAGE A3 TCR, 4-1BB, 5T4, adenocarcinoma antigen, alpha-fetoprotein, BAFF, B-lymphoma cell, C242 antigen, CA-125, carbonic anhydrase 9 (CA-IX), CCR4, CD152, CD200, CD22, CD19, CD22, CD123, CD221, CD23 (IgE receptor), CD28, CD30 (TNFRSF8), CD33, CD4, CD40, CD44, CD44v6, CD51, CD52, CD56, CD74, CD80, CS-1, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HGF, human scatter factor receptor kinase, IGF-1 receptor, IGF-I, IgGI, L1-CAM, IL-13, IL-6, insulin-like growth factor I receptor, integrin α5β1, integrin αvβ3, MORAb-009, MS4A1, MUC1, mucin CanAg, N-glycolylneuraminic acid, NPC-1C, PDGF-Rα, PDL192, phosphatidylserine, prostatic carcinoma cells, RANKL, RON, SCH 900105, SDC1, SLAMF7, TAG-72, tenascin C, TGF beta 2, TGF-β, TRAIL-R1, TRAIL-R2, tumour antigen CTAA16.88, VEGF-A, VEGFR-1, VEGFR2 or vimentin.

Preferably, the antigen-specific binding domain specifically binds to a tumour antigen. In a specific embodiment, the CAR comprises a single chain Fv that specifically binds CD44v6.

An exemplary antigen-specific targeting domain is a CD44v6-specific single-chain fragment (scFV) such as described in Casucci, M. et al. (Casucci, M. et al. (2013) Blood 122: 3461-72). Such a sequence is shown below:

MEAPAQLLFLLLLWLPDTTGEIVLTQSPATLSLSPGERATLSCSASSSIN
YIYWLQQKPGQAPRILIYLTSNLASGVPARFSGSGSGTDFTLTISSLEPE
DFAVYYCLQWSSNPLTFGGGTKVEIKRGGGGSGGGGSGGGGSGGGGSEVQ
LVESGGGLVKPGGSLRLSCAASGFTFSSYDMSWVRQAPGKGLEWVSTISS
GGSYTYYLDSIKGRFT ISRDNAKNSLYLQMNSLRAEDTAVYYCARQGLD
YWGRGTLVTVSS (SEQ ID NO: 17; CD44v6-specific
single-chain fragment (scFv))

In one embodiment, the CD44v6-specific single-chain fragment comprises at least 85%, 90%, 95%, 97%, 98% or 99% identity to SEQ ID NO: 17.

In a further preferred embodiment, the light chain variable region and the heavy chain variable region of the CD44v6-specific single chain fragment are connected to one another via a peptide linker having the following sequence GGGGSGGGGS (4GS2; SEQ ID NO: 36). Such CD44v6-specific single chain fragment (CD44v6-4GS2) has the following sequence:

(SEQ ID NO: 31)
MEAPAQLLFLLLLWLPDTTGEIVLTQSPATLSLSPGERATLSCSASSSIN
YIYWLQQKPGQAPRILIYLTSNLASGVPARFSGSGSGTDFTLTISSLEPE
DFAVYYCLQWSSNPLTFGGGTKVEIKRGGGGSGGGGSEVQLVESGGGLVK
PGGSLRLSCAASGFTFSSYDMSWVRQAPGKGLEWVSTISSGGSYTYYLDS
IKGRFTISRDNAKNSLYLQMNSLRAEDTAVYYCARQGLDYWGRGTLVTVS
S

### Co-Stimulatory Domain

The CAR of the invention may also comprise one or more co-stimulatory domains. The co-stimulatory domain may enhance cell proliferation, cell survival and development of memory cells.

Each co-stimulatory domain comprises the co-stimulatory domain of any one or more of, for example, members of the TNFR superfamily, CD28, CD137 (4-1 BB), CD134 (OX40), DapIO, CD27, CD2, CD5, ICAM-1, LFA-1, Lck, TNFR-1, TNFR-II, Fas, CD30, CD40 or combinations thereof. Co-stimulatory domains from other proteins may also be used with the CAR of the invention. Additional co-stimulatory domains will be apparent to the skilled person.

In one embodiment the transmembrane and co-stimulatory domain are both derived from CD28. In one embodiment the transmembrane and intracellular co-stimulatory domain comprise the sequence below:

FWVLVVVGGVLACYSLLVTVAFIIFWVRSKRSRLLHSDYMNMTPRRPOPT
RKHYQPYAPPRDFAAYRS (SEQ ID NO: 18; transmembrane
and intracellular portion of the human CD28
(UNIPROT: P10747, CD28 HUMAN, position 153-220))

In one embodiment, the transmembrane and intracellular signalling domain comprises at least 85%, 90%, 95%, 97%, 98% or 99% identity to SEQ ID NO: 18.

In one embodiment, the transmembrane domain of CD28 comprises the sequence:

(SEQ ID NO: 29)
FWVLVVVGGVLACYSLLVTVAFIIFWV

In one embodiment, the intracellular co-stimulatory domain of CD28 comprises the sequence:

(SEQ ID NO: 30)
RSERSRLLHSDYMNMTPRRPOPTREHYQPYAPPRDFAAYRS

### Intracellular Signalling Domain

The CAR of the invention may also comprise an intracellular signalling domain. The intracellular signalling domain may be cytoplasmic and may transduce the effector function signal and direct the cell to perform its specialised function. Examples of intracellular signalling domains include, but are not limited to, ζ chain of the T cell receptor or any of its homologues (e.g. η chain, FcεR1γ and β chains, MB1 (Igα) chain, B29 (Igβ) chain), CD3 polypeptides (Δ, δ and ε), syk family tyrosine kinases (e.g. Syk, ZAP 70), src family tyrosine kinases (e.g. Lck, Fyn, Lyn) and other molecules involved in T cell transduction, such as CD2, CD5 and CD28. The intracellular signalling domain may be human CD3 zeta chain, FcyRIII, FcsRI, cytoplasmic tails of Fc receptors, immunoreceptor tyrosine-based activation motif (ITAM) bearing cytoplasmic receptors or combinations thereof.

Preferably, the intracellular signalling domain comprises the intracellular signalling domain of human CD3 zeta chain.

In one embodiment the intracellular signalling domain of human CD3 zeta chain comprises the following sequence:

RVKFSRSADAPAYQQGQNQLYNELNLGRREEYDVLDKRRGRDPEMGGKPQ
RRKNPQEGLYNELQKDKMAEAYSEIGMKGERRRGKGHDGLYQGLSTATKD
TYDALHMQALPPR (SEQ ID NO: 20; UNIPROT: P20963,
CD3Z_HUMAN, positions 31-143)

In one embodiment, the intracellular signalling domain comprises at least 85%, 90%, 95%, 97%, 98% or 99% identity to SEQ ID NO: 20.

Additional intracellular signalling domains will be apparent to the skilled person and may be used in connection with alternate embodiments of the invention.

### Transmembrane Domain

The CAR of the invention may also comprise a transmembrane domain. The transmembrane domain may comprise the transmembrane sequence from any protein which has a transmembrane domain, including any of the type I, type II or type III transmembrane proteins. The transmembrane domain of the CAR of the invention may also comprise an artificial hydrophobic sequence. The transmembrane domains of the CARs of the invention may be selected so as not to dimerise. Additional transmembrane domains will be apparent to the skilled person.

Examples of transmembrane (TM) regions used in CAR constructs are: (a) the CD28 TM region (Pule et al. (2005) Mol. Ther. 12:933-41; Brentjens et al. (2007) CCR13:5426-35; Casucci et al. (2013) Blood 122:3461-72); (b) the OX40 TM region (Pule et al. (2005) Mol. Ther. 12:933-41); (c) the 41BB TM region (Brentjens et al. (2007) CCR13:5426-35); (d) the CD3 zeta TM region (Pule et al. (2005) Mol. Ther. 12:933-41; Savoldo, B. (2009) Blood 113:6392-402); (e) the CD8a TM region (Maher et al. (2002) Nat. Biotechnol. 20:70-5; Imai, C. (2004) Leukemia 18:676-84; Brentjens et al. (2007) CCR13: 5426-35; Milone et al. (2009) Mol. Ther. 17:1453-64).

In one embodiment, the transmembrane and intracellular signalling domain are both derived from CD28.

### Spacer Domain—Low Affinity Nerve Growth Factor Receptor (LNGFR)

The CAR of the invention may comprise an extracellular spacer domain. The extracellular spacer domain is attached to the antigen-specific targeting region and the transmembrane domain.

The CAR of the present invention may comprise an extracellular spacer which comprises at least part of the extracellular domain of human low affinity nerve growth factor receptor (LNGFR) or a derivative thereof.

LNGFR is not expressed on the majority of human haematopoietic cells, thus allowing quantitative analysis of transduced gene expression by immunofluorescence, with single cell resolution. Thus, fluorescence activated cell sorter analysis of expression of LNGFR may be performed in transduced cells to study gene expression. Further details on analysis using LNGFR may be found in Mavilio et al. (Mavilio et al. (1994) Blood 83: 1988-1997).

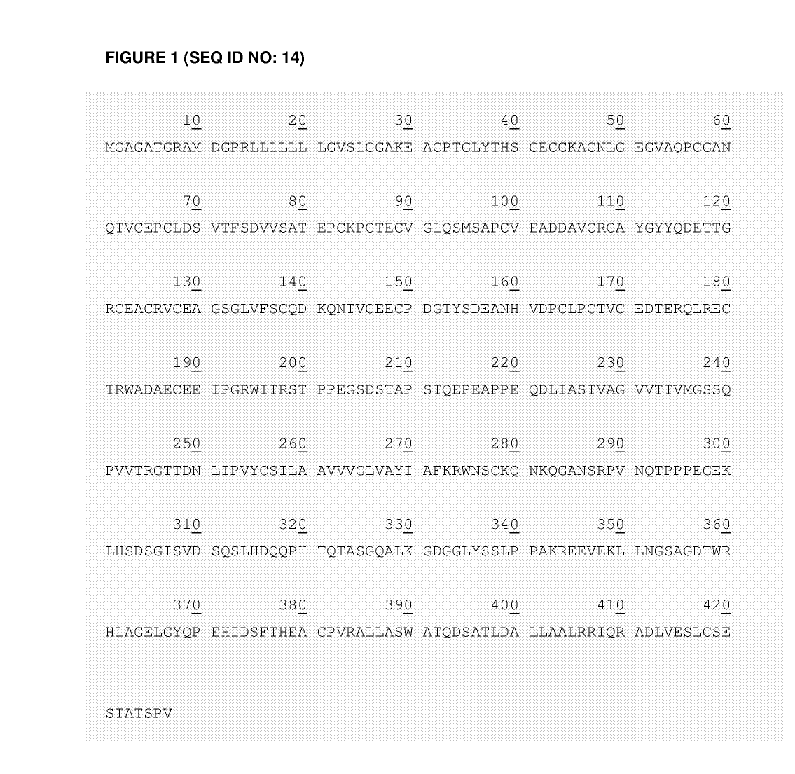

An example sequence of human LNGFR is shown in FIG. 1 (SEQ ID NO: 14).

In one embodiment, the invention makes use of a truncated LNGFR (also known as ΔLNGFR). Preferably, the LNGFR used in the invention is truncated in its intracytoplasmic domain. Such a truncation is described in Mavilio et al. (Mavilio et al. (1994) Blood 83: 1988-1997).

Preferably, the LNGFR spacer of the invention comprises at least part of the extracellular domain or a derivative thereof but lacks the intracellular domain of LNGFR. The extracellular domain may comprise amino acids 29-250 of LNGFR or a derivative thereof.

KEACPTGLYTHSGECCKACNLGEGVAQPCGANQTVCEPCLDSVTFSDVVS
ATEPCKPCTECVGLQSMSAPCVEADDAVCRCAYGYYQDETTGRCEACRVC
EAGSGLVFSCQDKQNTVCEECPDGTYSDEANHVDPCLPCTVCEDTERQLR
ECTRWADAECEEIPGRWITRSTPPEGSDSTAPSTQEPEAPPEQDLIASTV
AGVVTTVMGSSQPVVTRGTTDN (SEQ ID NO: 19;
extracellular domain of the human LNGFR
(UNIPROT: P08138, TNR16 HUMAN, positions 29-250))

Preferably the LNGFR lacks the signal peptide.

In one embodiment, the spacer comprises at least part of a protein having at least 85%, 90%, 95%, 96%, 97%, 98% or 99% identity to the extracellular domain of LNGFR (e.g. SEQ ID NO:19). In one embodiment, the spacer comprises at least part of a protein having at least 85%, 90%, 95%, 96%, 97%, 98% or 99% identity to amino acids 29-250 of the LNGFR protein.

LNGFR comprises 4 TNFR-Cys domains (TNFR-Cys 1, TNFR-Cys 2, TNFR-Cys 3 and TNFR-Cys 4). Sequences of the domains are exemplified below:

TNFR-Cys 1:
(SEQ ID NO: 9)
ACPTGLYTHSGECCKACNLGEGVAQPCGANQTVC
TNFR-Cys 2:
(SEQ ID NO: 10)
PCLDSVTFSDVVSATEPCKPCTECVGLQSMSAPCVEADDAVC
TNFR-Cys 3:
(SEQ ID NO: 11)
RCAYGYYQDETTGRCEACRVCEAGSGLVFSCQDKQNTVC
TNFR-Cys 4:
(SEQ ID NO: 12)
ECPDGTYSDEANHVDPCLPCTVCEDTERQLRECTRWADAEC

In one embodiment, the spacer comprises TNFR-Cys 1, 2 and 3 domains or fragments or derivatives thereof. In another embodiment, the spacer comprises the TNFR-Cys 1, 2, 3 and 4 domains or fragments or derivatives thereof.

In one embodiment the spacer comprises a sequence having at least 80%, 85%, 90%, 95%, 96%, 97%, 98%, 99% or 100% identity to TNFR-Cys 1 (SEQ ID NO: 9), a sequence having at least 80%, 85%, 90%, 95%, 96%, 97%, 98%, 99% or 100% identity to TNFR-Cys 2 (SEQ ID NO: 10), or a sequence having at least 80%, 85%, 90%, 95%, 96%, 97%, 98%, 99% or 100% identity to TNFR-Cys 3 (SEQ ID NO: 11). The spacer may further comprise a sequence having at least 80%, 85%, 90%, 95%, 96%, 97%, 98%, 99% or 100% identity to TNFR-Cys 4 (SEQ ID NO: 12).

Rather than comprise the full TNFR-Cys 4 domain, the spacer may comprise a TNFR-Cys 4 domain with the following amino acids deleted from said domain: NHVDPCLPCTVCEDTERQLRECTRW. In one embodiment, the NHVDPCLPCTVCEDTERQLRECTRW amino acids are replaced with the following amino acids ARA.

In one embodiment, the spacer lacks the LNGFR serine/threonine-rich stalk. In another embodiment the spacer comprises the LNGFR serine/threonine-rich stalk.

The spacer may comprise or consist of a sequence of SEQ ID NO: 1 or a sequence having at least 85%, 90%, 95%, 96%, 97%, 98% or 99% identity to SEQ ID NO: 1.

The spacer may comprise or consist of a sequence of SEQ ID NO: 3 or a sequence having at least 85%, 90%, 95%, 96%, 97%, 98% or 99% identity to SEQ ID NO: 3.

The spacer may comprise or consist of a sequence of SEQ ID NO: 5 or a sequence having at least 85%, 90%, 95%, 96%, 97%, 98% or 99% identity to SEQ ID NO: 5.

The spacer may comprise or consist of a sequence of SEQ ID NO: 7 or a sequence having at least 85%, 90%, 95%, 96%, 97%, 98% or 99% identity to SEQ ID NO: 7.

The spacer may confer properties to the CAR such that it allows for immunoselection of cells, preferably lymphocytes, expressing said CAR.

The CAR of the invention (preferably comprising the spacer referred to herein) preferably enables cells (e.g. lymphocytes) expressing the CAR to proliferate in the presence of cells expressing the antigen for which the CAR is designed.

The CAR of the invention (preferably comprising the spacer referred to herein) preferably enables cells (e.g. lymphocytes) expressing the CAR to mediate therapeutically significant anti-cancer effects against a cancer that the CAR is designed to target.

The CAR of the invention (preferably comprising the spacer referred to herein) is preferably suitable for facilitating immunoselection of cells transduced with said CAR.

The CAR of the invention comprising the LNGFR-based spacer avoids activation of unwanted and potentially toxic off-target immune responses and allows CAR-expressing cells to persist in vivo without being prematurely cleared by the host immune system.

The present invention also encompasses the use of variants, derivatives, homologues and fragments of the spacer elements described herein.

### Peptide

The term “peptide” as used herein includes polypeptides and proteins. The term “polypeptide” includes single-chain polypeptide molecules as well as multiple-polypeptide complexes where individual constituent polypeptides are linked by covalent or non-covalent means. The term “polypeptide” includes peptides of two or more amino acids in length, typically having more than 5, 10, 20, 30, 40, 50 or 100 amino acids.

Peptides may comprise non-naturally-occurring amino acids, which have been modified, for example, to reduce immunogenicity, to increase circulatory half-life in the body of the patient, to enhance bioavailability, and/or to enhance efficacy and/or specificity.

A number of approaches have been used to modify peptides for therapeutic application. One approach is to link the peptides or proteins to a variety of polymers, such as polyethylene glycol (PEG) and polypropylene glycol (PPG) (see, for example, U.S. Pat. Nos. 5,091,176, 5,214,131 and 5,264,209).

Replacement of naturally-occurring amino acids with a variety of uncoded or modified amino acids such as D-amino acids and N-methyl amino acids may also be used to modify peptides.

Another approach is to use bifunctional crosslinkers, such as N-succinimidyl 3-(2 pyridyldithio) propionate, succinimidyl 6-[3-(2 pyridyldithio) propionamido] hexanoate and sulfosuccinimidyl 6-[3-(2 pyridyldithio) propionamido]hexanoate (see, for example, U.S. Pat. No. 5,580,853).

The active conformation of the peptide may be stabilised by a covalent modification, such as cyclisation, or by incorporation of gamma-lactam or other types of bridges. For example, side chains can be cyclised to the backbone so as create a L-gamma-lactam moiety on each side of the interaction site. See, generally, Hruby et al. (1992) “Applications of Synthetic Peptides” in Synthetic Peptides: A User's Guide: 259-345, W.H. Freeman & Co. Cyclisation can also be achieved, for example, by formation of cysteine bridges, coupling of amino and carboxy terminal groups of respective terminal amino acids, or coupling of the amino group of a lysine residue or a related homologue with a carboxy group of aspartic acid, glutamic acid or a related homologue. Coupling of the alpha-amino group of a polypeptide with the epsilon-amino group of a lysine residue, using iodoacetic anhydride, can be also undertaken (see, for example, Wood et al. (1992) Int. J. Peptide Protein Res. 39: 533-39).

A further technique for improving the properties of therapeutic peptides is to use non-peptide peptidomimetics. A wide variety of useful techniques may be used to elucidate the precise structure of a peptide. These techniques include amino acid sequencing, X-ray crystallography, mass spectrometry, nuclear magnetic resonance spectroscopy, computer-assisted molecular modelling, peptide mapping and combinations thereof. Structural analysis of a peptide generally provides a large body of data, which comprises the amino acid sequence of the peptide as well as the three-dimensional positioning of its atomic components. From this information, non-peptide peptidomimetics may be designed that have the required chemical functionalities for therapeutic activity, but are more stable, for example less susceptible to biological degradation. An example of this approach is provided in U.S. Pat. No. 5,811,512.

Techniques for chemically synthesising therapeutic peptides of the invention are described in the above references and also reviewed by Borgia et al. (Borgia et al. (2000) TibTech 18: 243-251) and described in detail in the references contained therein.

### Variants, Derivatives and Fragments

In addition to the specific proteins, peptides and polynucleotides mentioned herein, the invention also encompasses the use of variants, derivatives and fragments thereof.

The terms “variant” or “derivative” in relation to proteins or polypeptides of the invention includes any substitution of, variation of, modification of, replacement of, deletion of and/or addition of one (or more) amino acids from or to the sequence, providing that the resultant protein or polypeptide retains the desired function.

Typically, amino acid substitutions may be made, for example from 1, 2 or 3 to 10 or 20 substitutions provided that the modified sequence retains the required activity or ability. Amino acid substitutions may include the use of non-naturally occurring analogues.

Proteins or polypeptides used in the invention may also have deletions, insertions or substitutions of amino acid residues, which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R H |
| AROMATIC | | F W Y |

Fragments typically refer to a selected region of the polypeptide or polynucleotide that is of interest functionally. “Fragment” thus refers to an amino acid sequence that is a portion of a full length polypeptide or a nucleic acid sequence that is a portion of a full-length polynucleotide. Since fragments are of interest functionally e.g. retain the desired functionality, they will therefore exclude e.g. a single amino acid or a single nucleic acid.

The peptides used in the invention are typically made by recombinant means. However, they may also be made by synthetic means using techniques well known to the skilled person, such as solid phase synthesis. Various techniques for chemically synthesising peptides are reviewed by Borgia et al. (Borgia et al. (2000) TibTech 18: 243-251) and described in detail in the references contained therein.

The derivative may be a homologue. The term “homologue” as used herein means an entity having a certain homology with the wild type amino acid sequence and/or the wild type nucleotide sequence. The term “homology” can be equated with “identity”.

A homologous sequence may include an amino acid sequence which may be at least 50%, 55%, 65%, 75%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 65%, 75%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percent homology or identity between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percent homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8).

Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

Such derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

### Polynucleotides

Polynucleotides encoding the TNF conjugates and chimeric antigen receptor (CAR) may be used in the present invention.

Polynucleotides of the invention may comprise DNA or RNA. They may be single-stranded or double-stranded. It will be understood by a skilled person that numerous different polynucleotides can encode the same polypeptide as a result of the degeneracy of the genetic code. In addition, it is to be understood that the skilled person may, using routine techniques, make nucleotide substitutions that do not affect the polypeptide sequence encoded by the polynucleotides of the invention to reflect the codon usage of any particular host organism in which the polypeptides of the invention are to be expressed.

The polynucleotides may be modified by any method available in the art. Such modifications may be carried out in order to enhance the in vivo activity or lifespan of the polynucleotides of the invention.

Polynucleotides such as DNA polynucleotides may be produced recombinantly, synthetically or by any means available to the skilled person. They may also be cloned by standard techniques.

Longer polynucleotides will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

### Codon Optimisation

The polynucleotides used in the present invention may be codon-optimised. Codon optimisation has previously been described in WO 1999/041397 and WO 2001/079518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

### Vectors

A vector is a tool that allows or facilitates the transfer of an entity from one environment to another. In accordance with the invention, and by way of example, some vectors used in recombinant nucleic acid techniques allow entities, such as a segment of nucleic acid (e.g. a heterologous DNA segment, such as a heterologous cDNA segment), to be transferred into a target cell. The vector may serve the purpose of maintaining the heterologous nucleic acid (e.g. DNA or RNA) within the cell, facilitating the replication of the vector comprising a segment of nucleic acid or facilitating the expression of the protein encoded by a segment of nucleic acid.

Vectors may be non-viral or viral. Examples of vectors used in recombinant nucleic acid techniques include, but are not limited to, plasmids, chromosomes, artificial chromosomes and viruses. The vector may also be, for example, a naked nucleic acid (e.g. DNA or RNA). In its simplest form, the vector may itself be a nucleotide of interest.

In one aspect, the invention provides a vector comprising a polynucleotide of the invention.

The vectors used in the invention may be, for example, plasmid or viral vectors and may include a promoter for the expression of a polynucleotide and optionally a regulator of the promoter.

### Viral Vectors

In a preferred embodiment, the vector used in the invention is a viral vector. Preferably, the viral vector is in the form of a viral vector particle.

The viral vector may be, for example, an adeno-associated viral (AAV), retroviral, lentiviral or adenoviral vector.

### Cells

The products and methods of the invention relate to cells, which comprise a chimeric antigen receptor (CAR) of the invention. Such cells may be cells genetically engineered to express the CAR of the invention.

Cells which may comprise and express the CARs of the invention include, but are not limited to, lymphocytes such as natural killer cells and/or T cells, including naive T cells, stem cell memory T cells, central memory T cells, effector memory T cells and/or haematopoietic stem cells and/or cells capable of giving rise to therapeutically relevant progeny.

In one embodiment, the cells are autologous cells.

By way of example, individual T cells of the invention may be CD4+/CD8−, CD4−/CD8+, CD4−/CD8− or CD4+/CD8+. The T cells may be a mixed population of CD4+/CD8− and CD4−/CD8+ cells or a population of a single clone.

Genetically modified cells may be produced by stably transfecting or transducing cells with DNA encoding the CAR of the invention.

Various methods produce stable transfectants, which express the CARs of the invention. In one embodiment, a method of stably transfecting and redirecting cells is by electroporation using naked DNA. By using naked DNA, the time required to produce redirected cells may be significantly reduced. Additional methods to genetically engineer cells using naked DNA encoding the CAR of the invention include, but are not limited to, chemical transformation methods (e.g. using calcium phosphate, dendrimers, liposomes and/or cationic polymers), non-chemical transformation methods (e.g. electroporation, optical transformation, gene electrotransfer and/or hydrodynamic delivery) and/or particle-based methods (e.g. impalefection, using a gene gun and/or magnetofection). The transfected cells demonstrating presence of a single integrated un-rearranged vector and expression of the CAR may be expanded ex vivo. In one embodiment, the cells selected for ex vivo expansion are CD8+ and demonstrate the capacity to specifically recognise and lyse antigen-specific target cells.

Viral transduction methods may also be used to generate redirected cells, which express the CAR of the invention.

Stimulation of the T cells by an antigen under proper conditions results in proliferation (expansion) of the cells and/or production of IL-2. The cells comprising the CAR of the invention will expand in number in response to the binding of one or more antigens to the antigen-specific targeting regions of the CAR. The invention also provides a method of making and expanding cells expressing a CAR. The method may comprise transfecting or transducing the cells with the vector expressing the CAR after stimulating the cells with: (a) polyclonal stimuli such as cell-free scaffolds, preferably optimally-sized beads, containing at least an activating polypeptide, preferably an antibody, specific for CD3 and an activating polypeptide, preferably an antibody, specific for CD28; (b) tumour cells expressing the target antigen; (c) natural artificial antigen presenting cells, and culturing them with cytokines including IL-2, IL-7, IL-15, IL-21 alone or in combination.

### Pharmaceutical Formulations

The present invention also provides a pharmaceutical composition for treating an individual wherein the composition comprises a therapeutically effective amount of the pharmaceutical product of the present invention.

The pharmaceutical composition may be for human or animal usage. Typically, a physician will determine the actual dosage which will be most suitable for an individual subject and it will vary with the age, weight and response of the particular individual.

The composition may optionally comprise a pharmaceutically acceptable carrier, diluent, excipient or adjuvant. The choice of pharmaceutical carrier, diluent or excipient can be selected with regard to the intended route of administration and standard pharmaceutical practice. The pharmaceutical compositions may comprise as (or in addition to) the carrier, diluent or excipient any suitable binder(s), lubricant(s), suspending agent(s), coating agent(s), solubilising agent(s) and other carrier agents that may aid or increase entry into the target site (such as, for example, a lipid delivery system). Suitable carriers and diluents include isotonic saline solutions, for example phosphate-buffered saline. Details of excipients may be found in The Handbook of Pharmaceutical Excipients, 2nd Edn, Eds Wade & Weller, American Pharmaceutical Association.

Where appropriate, the pharmaceutical compositions can be administered by any one or more of inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intracavernosally, intravenously, intramuscularly or subcutaneously. For parenteral administration, the compositions may be best used in the form of a sterile aqueous solution, which may contain other substances, for example enough salts or monosaccharides to make the solution isotonic with blood. For buccal or sublingual administration, the compositions may be administered in the form of tablets or lozenges, which can be formulated in a conventional manner.

Formulations for parenteral administration comprise injectable solutions or suspensions and liquids for infusions. For the preparation of the parenteral forms, an effective amount of the active ingredient will be dissolved or suspended in a sterile carrier, optionally adding excipients such as solubilisers, isotonicity agents, preservatives, stabilisers, emulsifiers or dispersing agents, and it will be subsequently distributed in sealed vials or ampoules.

The routes of administration and dosage regimens described are intended only as a guide since a skilled practitioner will be able to determine readily the optimum route of administration and dosage regimens for any particular patient and condition.

### Subject

As used herein, the term “subject” refers to an organism, including, for example, an animal. An animal includes, but is not limited to, a human, a non-human primate, a horse, a pig, a goat, a cow, a rodent, such as, but not limited to, a rat or a mouse, or a domestic pet, such as, but not limited to, a dog or a cat.

Preferably, the subject is a mammal, preferably human.

### Method of Treatment

The pharmaceutical product and compositions of the invention may be used in therapeutic treatment.

It is to be appreciated that all references herein to treatment include curative, palliative and prophylactic treatment.

The patient treated to be is preferably a human patient, although it is to be understood that the principles of the invention indicate that the invention is effective with respect to all mammals, which are intended to be included in the term “patient”. In this context, a mammal is understood to include any mammalian species in which treatment of diseases associated with cancer is desirable, particularly agricultural and domestic mammalian species.

The pharmaceutical product or pharmaceutical composition of the present invention may be used to treat or prevent cancer including but not limited to melanoma, cancer of the lung, pancreas, breast, colon, head, neck, prostate, larynx, ovary or brain. Preferably, the cancer comprises a solid tumour. Preferably, the cancers to be treated express the CD44v6 antigen (Casucci, M. et al. (2013) Blood 122: 3461-3472).

The pharmaceutical product and pharmaceutical compositions of the invention can be used in combined, separated or sequential preparations, also with other diagnostic or therapeutic substances, such as, but not limited to, doxorubicin, melphalan, cis-platin, gemcitabine and taxol.

The efficacy of treatment of a tumour may be assessed by any of various parameters well known in the art. This includes, but is not limited to, determinations of a reduction in tumour size, determinations of the inhibition of the growth, spread, invasiveness, vascularisation, angiogenesis and/or metastasis of a tumour, determinations of the inhibition of the growth, spread, invasiveness and/or vascularisation of any metastatic lesions, and/or determinations of an increased delayed type hypersensitivity reaction to tumour antigen. The efficacy of treatment may also be assessed by the determination of a delay in relapse or a delay in tumour progression in the subject or by a determination of survival rate of the subject, for example, an increased survival rate at one or five years post treatment.

### EXAMPLES

### Example 1

### Preparation of Murine NGR-TNF

NGR-mTNF expression was induced in transformed BL21 (ID3) Escherichia coli (Novagen, Podenzano, PC, Italy) using 1 mM IPTG (Sigma-Aldrich, St Louis, Mo., USA). Bacterial homogenate was clarified by flocculation with polyethyleneimine (Sigma-Aldrich), and soluble NGR-mTNF was purified by three-stage chromatography: ion-exchange chromatography using Q-Sepharose XL (GE Healthcare, Milan, Italy), mixed-mode chromatography using Capto Adhere (GE Healthcare) and ion-exchange chromatography using Q-Sepharose HP (GE Healthcare) in denaturing conditions. The endotoxin content of the purified NGR-mTNF, measured by the quantitative chromogenic Limulus amebocyte lysate test (BioWhittaker, Lonza, Walkersville, Md., USA), was 0.12 U/μg¹.

### Preparation of Human NGR-TNF

Human recombinant NGR-TNF (consisting of human TNF1-157 fused with the C terminus of CNGRCG) was prepared by recombinant DNA technology and purified essentially as described for murine TNF and NGR-TNF.

### NGR-TNF in the Clinic

As of 18 Nov. 2014, 1,197 patients had been enrolled in a total of 16 completed or ongoing clinical trials. Completed or ongoing trials confirmed the favourable tolerability profile of NGR-hTNF, as well as a significant clinical activity in terms of overall survival, progression-free survival and disease control rates, including disease stabilisations, partial responses (defined as any reduction of a tumour lesion equal to at least 30% of the largest diameter of the same), and one case of complete response (i.e. tumour eradication) observed in liver cancer.

Data collected in Phase I trials provided the rationale for evaluating NGR-hTNF at low doses as a single agent. Those results showed that NGR-hTNF was able to induce disease stabilisation in a significant portion of patients treated, regardless of the specific tumour type and without producing significant toxicity.

The Phase II clinical trials of NGR-hTNF as a single agent by using the optimal biological dose of 0.8 μg/m², defined in a Phase I trial (NGR002) exploring the low-dose range, (Gregorc, et al. (2010) European Journal of Cancer 46: 198-206). Among all doses tested, NGR-hTNF induced the highest frequency and longest duration of disease stabilisation at the dose of 0.8 μg/m², along with a significant anti-vascular effect, as shown by dynamic contrast-enhanced magnetic resonance imaging (DCE-MRI).

Indications selected were malignant pleural mesothelioma, colorectal cancer and liver cancer. The primary objective was progression-free survival. Results of these trials are summarised below.
Malignant pleural mesothelioma—This multicentre, non-randomised trial started in May 2007 and recruited 57 patients with disease progression after treatment with apemetrexed-based chemotherapy. NGR-hTNF was thus tested as second-line therapy, a line of therapy for which there is neither a drug with regulatory approval, nor a widely-accepted chemotherapy available. 43 patients were treated every three weeks, while 14 patients received NGR-hTNF once a week. The first positive results of the trial were published in 2010 (Gregorc, et al. (2010) Journal of Clinical Oncology 28: 2604-2611), a long-term follow-up was disclosed at the ASCO annual meeting 2012 (Rossoni, et al. Poster session ASCO 2012 (abstract in JCO 2012 (Vol 30, No 15, May 20 Supplement), 2012: 7076)).
Colorectal cancer—This multicentre, non-randomised trial started in January 2007 and recruited 46 patients affected by refractory disease (i.e. patients having undergone a median of three prior lines of treatment due to resistance to treatment). The long-term results of the trial (Santoro, et al. (2010) European Journal of Cancer 46: 2746-2752) showed a median overall survival of 13.1 months, while the median overall survival reported for the best supportive care—i.e. the only option left after the failure of standard lines of therapy—was of 4.6-6.0 months (Journal of Clinical Oncology 2007: 1658-64; Journal of Clinical Oncology 2008: 1626-34).
Liver cancer—This multicentre, non-randomised trial started in February 2007 and recruited 40 pre-treated patients. 27 patients were treated every three weeks, while 13 patients were treated once a week. Results of the trial were published in 2010 (Santoro, et al. (2010) Journal of Cancer 103: 837-844) and showed a disease control rate of 30% maintained for a median of 4.3 months and a median overall survival time of 8.9 months. The complete tumour eradication, lasting since May 2008, was obtained in one patient refractory to sorafenib; in another patient, a partial tumour regression lasting for 5.5 months was observed.

In April 2010, a randomised, double-blind, placebo-controlled, international multicentre Phase III trial started and was carried out in Europe, the United States, Canada and Egypt for the treatment of malignant pleural mesothelioma. This is a placebo-controlled trial designed to evaluate drug efficacy on a large patient population randomly assigned (randomised) into two groups. One group received the drug, while the other received a placebo (a pharmaceutical preparation with identical appearance to the drug, but drug-free and consisting only of innocuous substances). Neither the patient nor the physician (double-blind) were aware of which group each patient was assigned to.

The trial recruited 400 adult patients affected by malignant pleural mesothelioma with disease progression after the standard pemetrexed-based chemotherapy. The trial investigated the administration of NGR-hTNF plus the “Best Investigator's Choice” (BIC), versus placebo plus the same BIC, where the BIC may include either supportive care alone or supportive care combined with one chemotherapeutic agent chosen among doxorubicin, gemcitabine or vinorelbine. The randomisation ratio was 1:1 between NGR-hTNF and placebo. Before randomisation, investigators decided for each patient if he/she was a candidate for either supportive care alone or in combination with chemotherapy; patients were then randomly assigned to either of the two treatment arms by specific stratification factors. NGR-hTNF was administered intravenously as a 1-hour infusion at 0.8 μg/m² once a week, until disease progression; the placebo followed the same administration schedule in the control arm. BIC was delivered according to institutional and literature guidelines, and chemotherapy was administered as per standard clinical practice. The primary objective of the trial was overall survival duration. Secondary objectives included progression-free survival extension, disease control, safety profile and patient quality of life.

As reported in regulated information published on 5 May and 3 Jun. 2014, although not meeting its primary endpoint of overall survival in the entire patient population, the trial showed a statistically significant (unstratified p=0.02; stratified p=0.01) 40% improvement of overall survival in patients with poorer prognosis, who had progressed during or shortly after first-line chemotherapy. These patients represent 50% of the entire patient population and were identified by a pre-specified analysis based on prior treatment-free interval (TFI), i.e. the time elapsed between the end of first line and the beginning of second line treatment.

### Example 2

An object of the invention is to define an appropriate therapeutic combination scheme that promotes the extravasation of T cells genetically engineered to express a chimeric antigen receptor (CAR) and, at the same time, creates the most appropriate physiological conditions to improve the anti-tumour effect of such cells. The effective therapeutic scheme will preferably assure that CAR-transduced T-cells:
(a) arrive proximally to the site of the solid tumour that expresses the selected tumour antigen; and
(b) find the physiological conditions in which the appropriate activation status of the cells can be maintained and the CAR signalling mechanism responsible for anti-tumour effect can be effectively activated.

With this aim, T cells transduced with a CAR are co-administered with the recombinant protein NGR-TNF (a CNGRCG fusion with the N-terminus of TNF). Different therapeutic windows associated with different therapeutic effects may be exploited. NGR-TNF binding to tumour vessels is associated with a transient improvement of vessel permeability (WO 2003/093478). In order to exploit this transient effect, CAR transduced cells may be infused shortly after NGR-TNF administration. This almost subsequent administration allows the avoidance of activation of potential counter regulatory mechanisms that may be associated with the administration of the NGR-TNF.

The transient effect may be exploited by administering the T cells genetically engineered to express a CAR in the period immediately after the administration of NGR-TNF. For example, this therapeutic scheme may comprise a single administration of NGR-TNF followed by the infusion of CAR transduced T cells after 1, 2 or 4 hours.

In addition, this therapeutic scheme may comprise multiple administrations of NGR-TNF, preferably three doses of NGR-TNF administered in one week, followed by infusion of CAR transduced T-cells after 1, 2 or 4 hours.

Administration of NGR-TNF has also been associated with normalisation of tumour vessels (Porcellini, S. et al. (2015) Oncoimmunology 4: e1041700). This effect does not appear to be transient, but it can be observed for a number of days after administration of NGR-TNF. In order to exploit this therapeutic window, CAR transduced cells may be infused after a relatively long period following NGR-TNF administration. The advantage of exploiting this therapeutic window is that the normalisation of tumour vessels is consolidated, thus promoting the best physiological conditions to obtain an effective immune response activated through CAR signalling.

Accordingly, this therapeutic scheme may comprise a single administration of NGR-TNF followed by infusion of CAR transduced T-cells after 12, 24, 48, 72 or 96 hours, or after 1 week.

In addition, the therapeutic scheme may comprise multiple administrations of NGR-TNF, preferably three doses of NGR-TNF administered in one week, followed by infusion of CAR transduced T-cells after 12, 24, 48, 72 or 96 hours, or 1 week.

Animal models will be used in the study. In order to better understand the immunological response achieved with the therapeutic scheme under analysis, C57BL/6 and BALB/c immunocompetent mice will be used. After a period of acclimatisation in the animal house mice destined to experience tumour growth will be inoculated subcutaneously (s.c.) with murine tumour cells (100-300 μL/mouse) (using a fine needle, 27G). As a “model” tumour, cell lines will be used to induce tumours in mice of the C57BL/6 and BALB/c strain. The inoculated tumour cell lines will be tested for mycoplasma prior to infusion in animals in order to avoid contamination of the enclosure.

Following inoculation of tumour cells, the health of the mice will be monitored daily for the appearance of toxic effects through the observation of clinical symptoms (e.g. difficulty in movement, appearance of the hump, ruffling the hair and/or macroscopic appearance of tumours) and detection of body weight. Mice carrying a visible tumour will be sacrificed when the tumour reaches a mean diameter of 1.0 cm, unless the mice exhibit clinical signs that require immediate intervention.

When the tumour is present, the animals will be treated intraperitoneally (i.p.) with an NGR-TNF single dose or in multiple treatments (three times weekly). After 2 or 72 hours following the last treatment with NGR-TNF, mice will be infused intravenously with T lymphocytes expressing the CD44v6-CAR-T using a fine needle syringe (29G).

### Example 3

### Generation of LNGFR-Spaced CD44v6-CAR.28z Constructs

Sequences of the LNGFR-based spacers may be derived from the extracellular portion of the low affinity nerve growth factor receptor (LNGFR), excluding the signal peptide (P08138, TNR16_HUMAN).

The wild-type long (NWL) design contains both the four TNFR cysteine-rich domains and the serine/threonine-rich stalk. The wild-type short (NWS) design comprises only the four TNFR cysteine-rich domains. The mutated long (NML) design contains the four TNFR cysteine-rich domains, the serine/threonine-rich stalk and includes a specific modification in the fourth domain to avoid binding to NGF (Yan et al. (1991) J. Biol. Chem. 266:12099-104). The mutated short (NMS) design contains only the four TNFR cysteine-rich domains including the specific modification in the fourth domain.

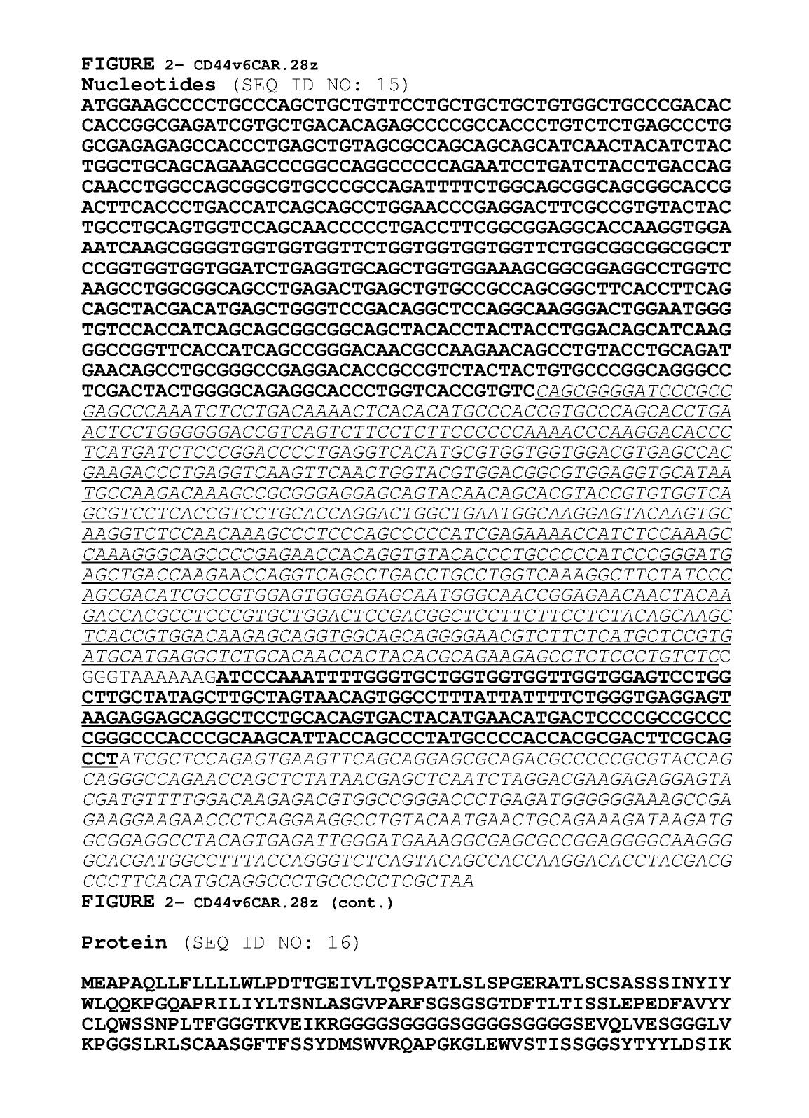

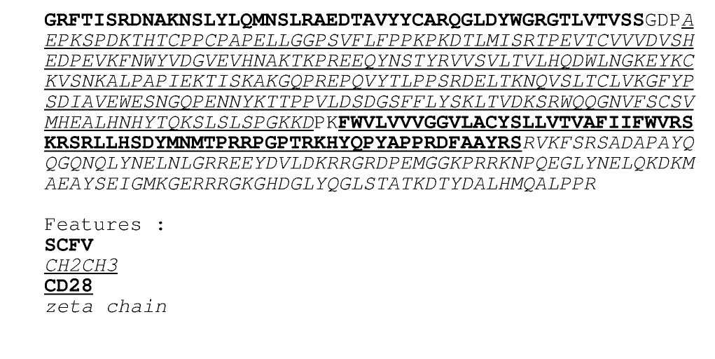

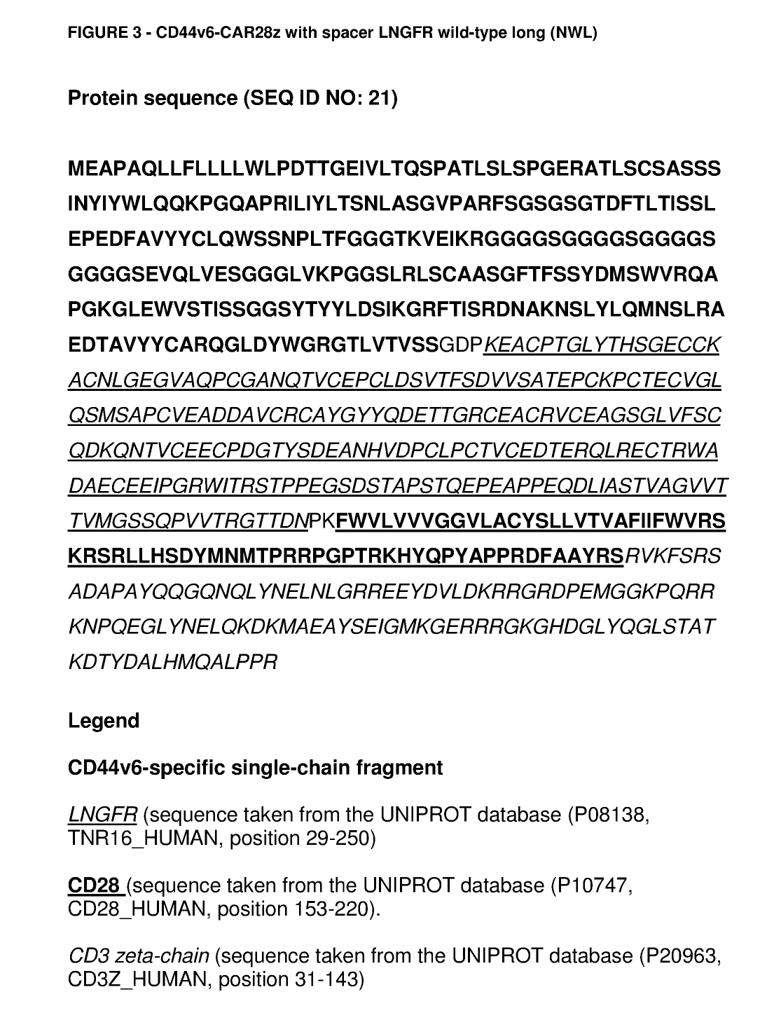

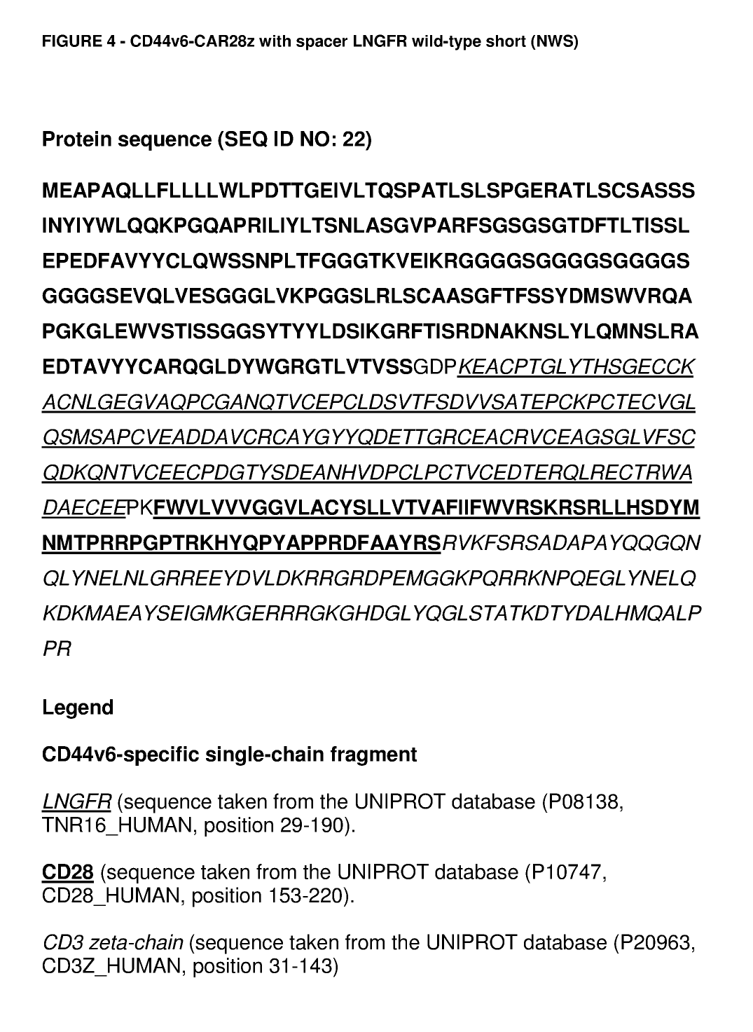

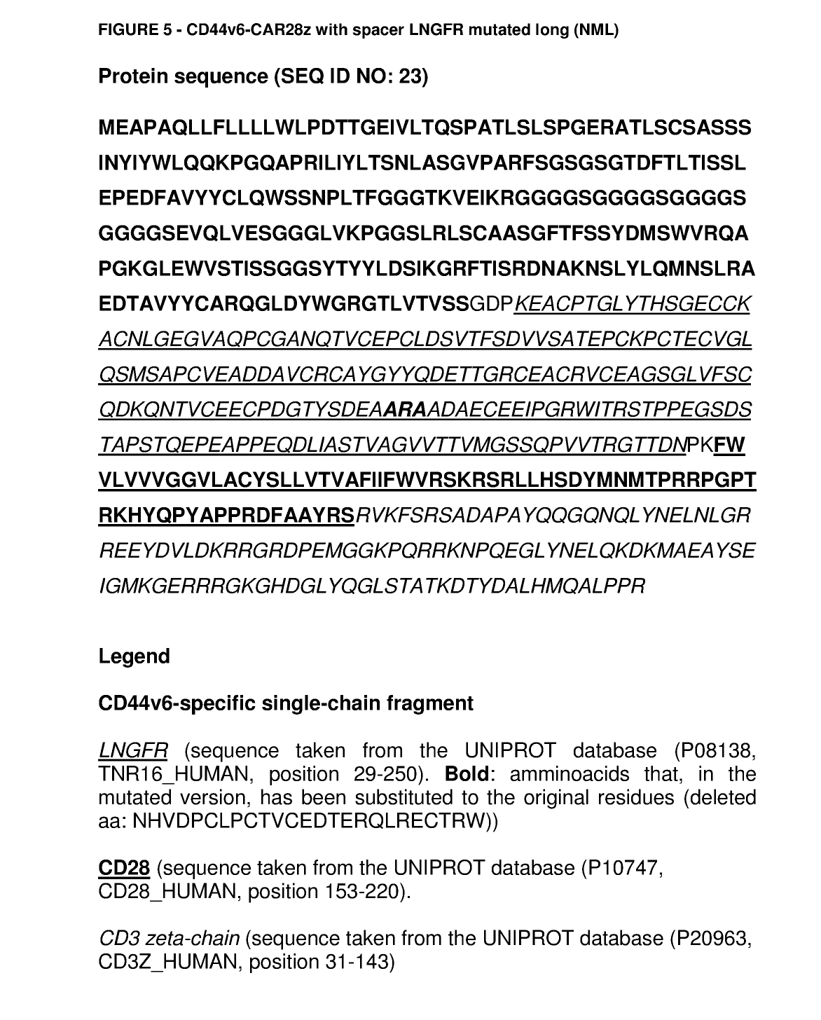

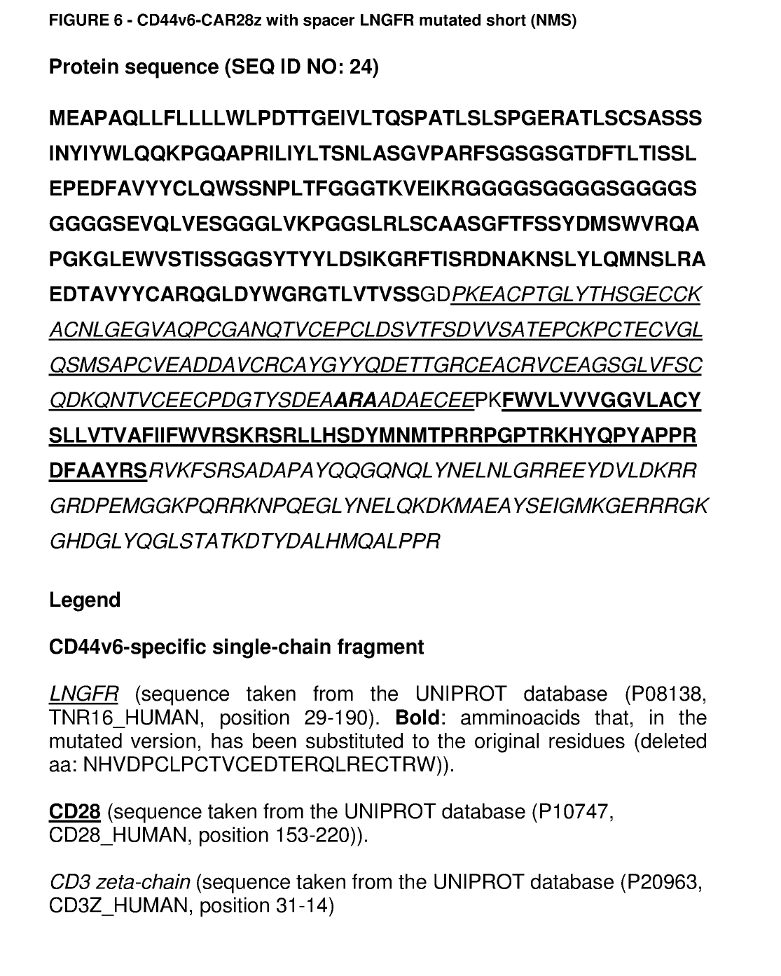

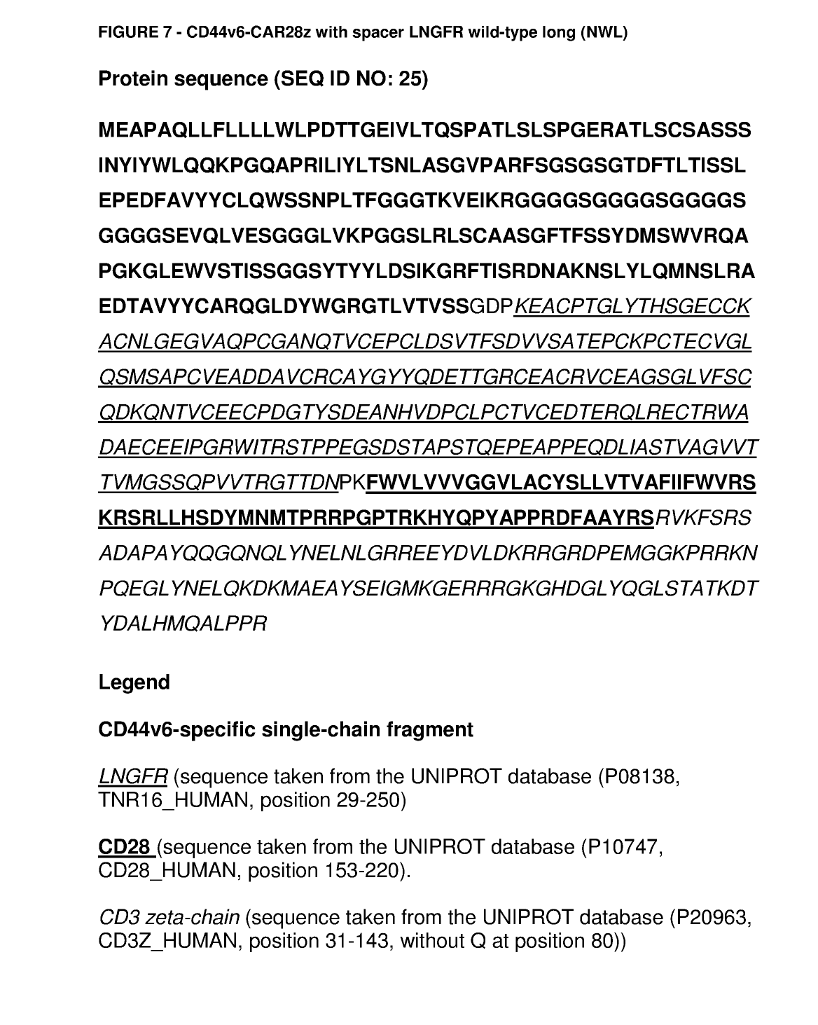

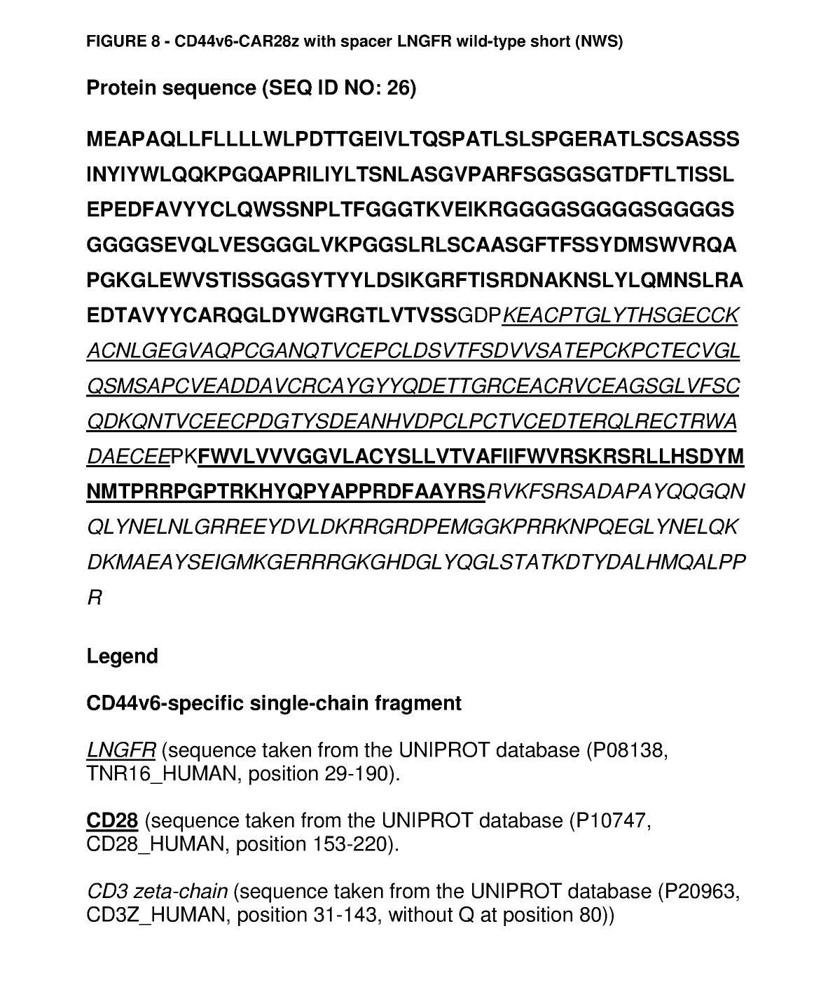

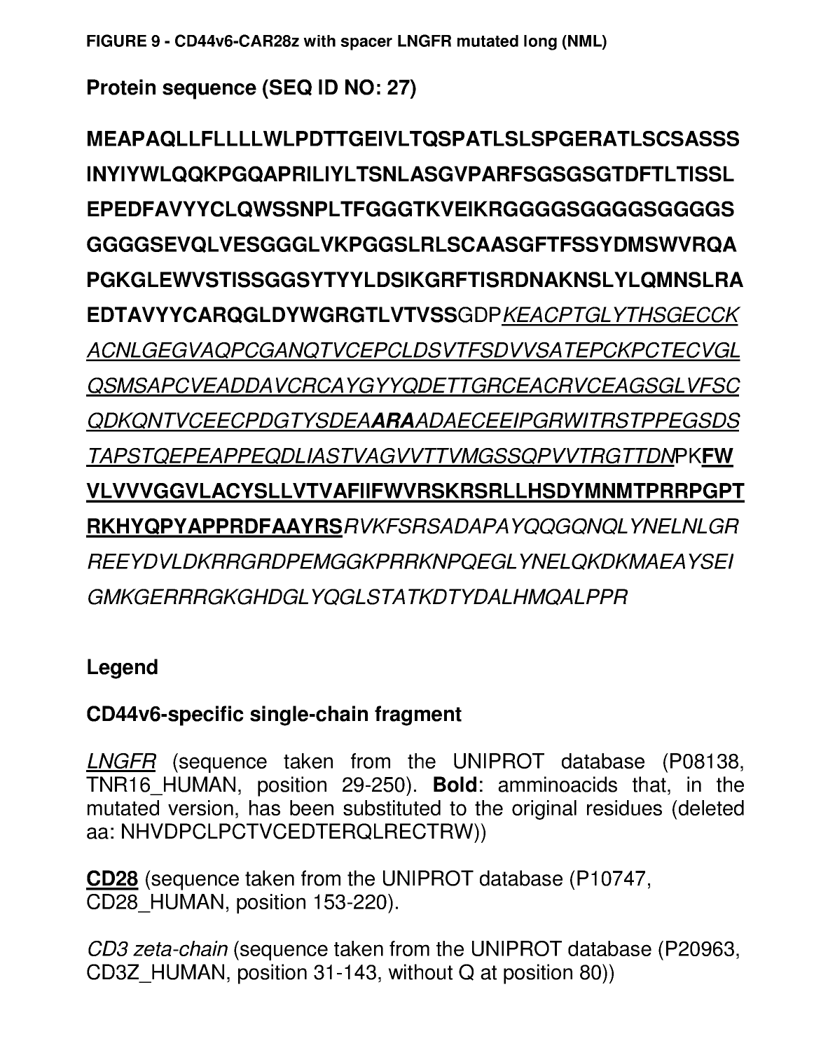

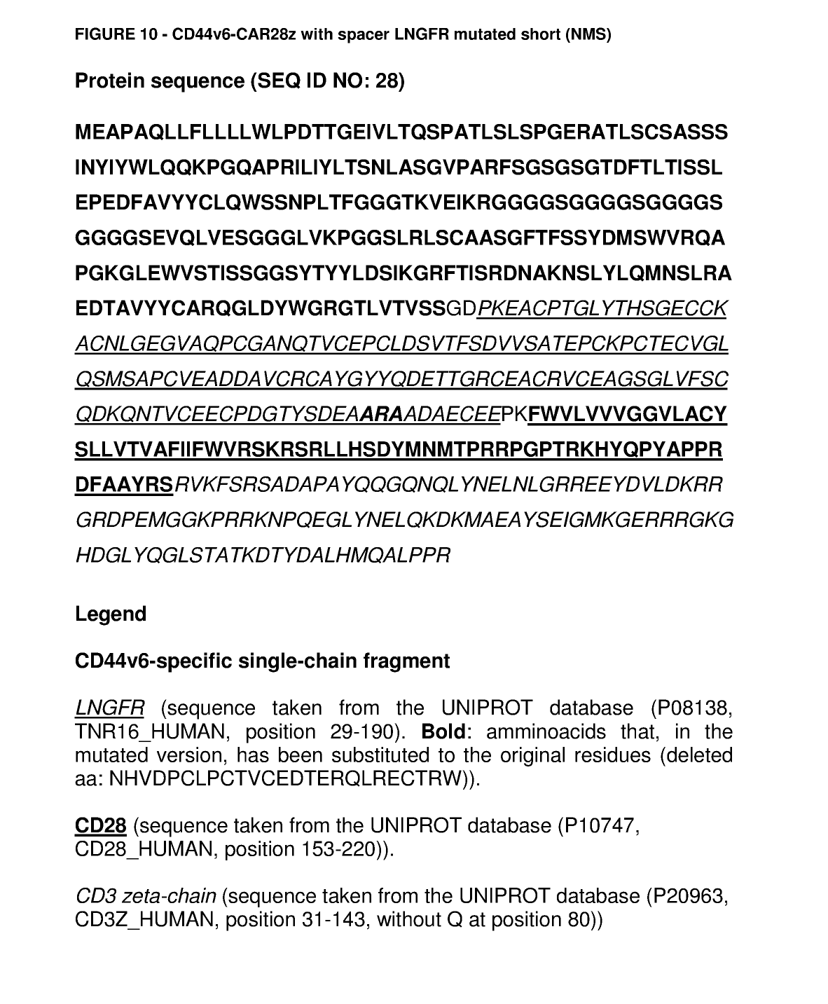

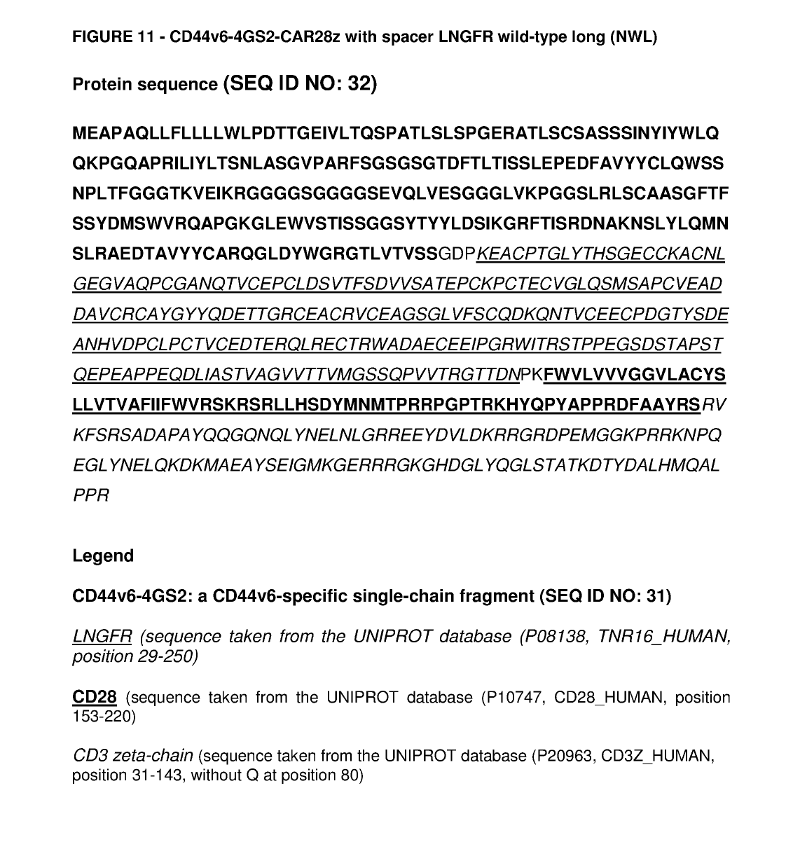

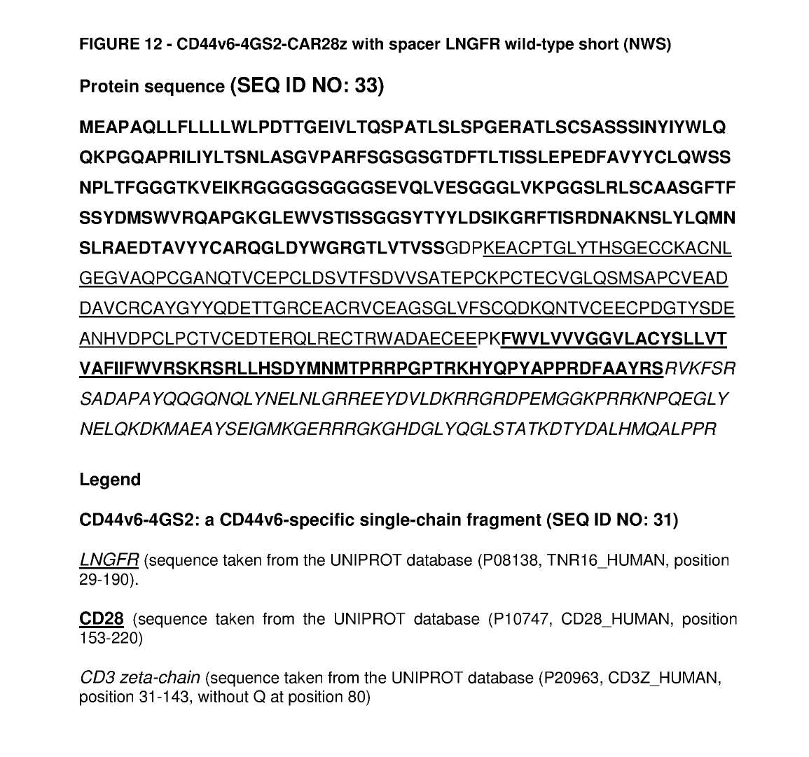

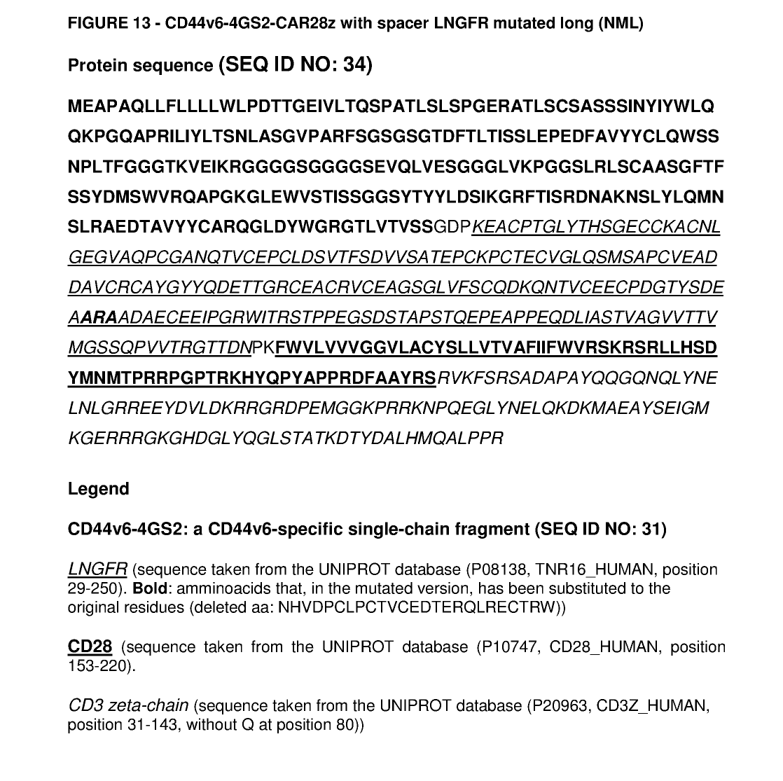

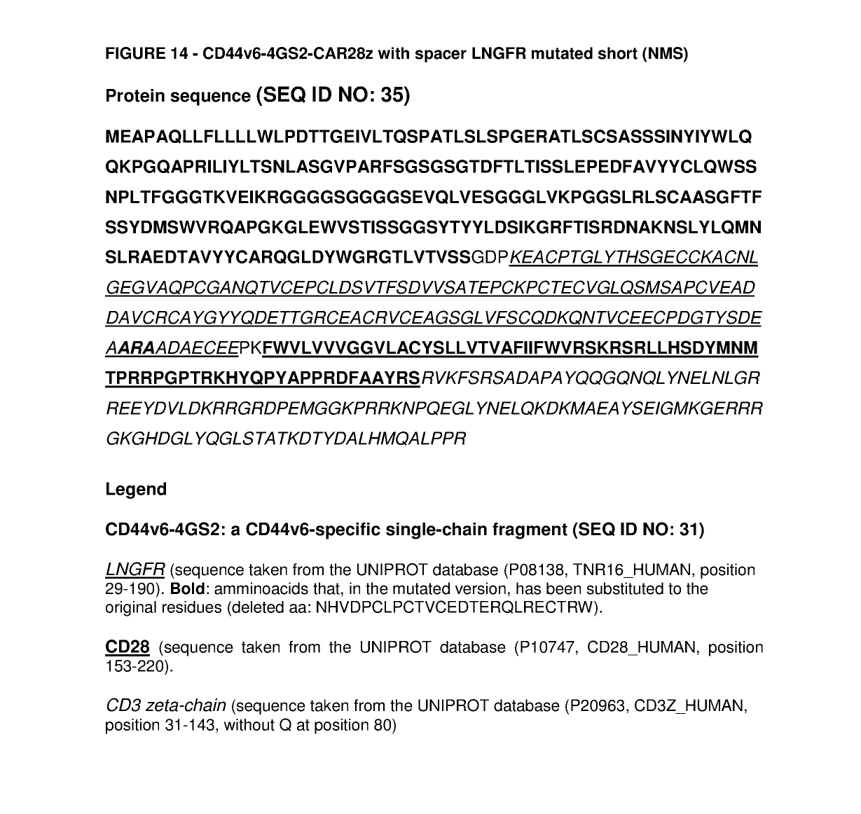

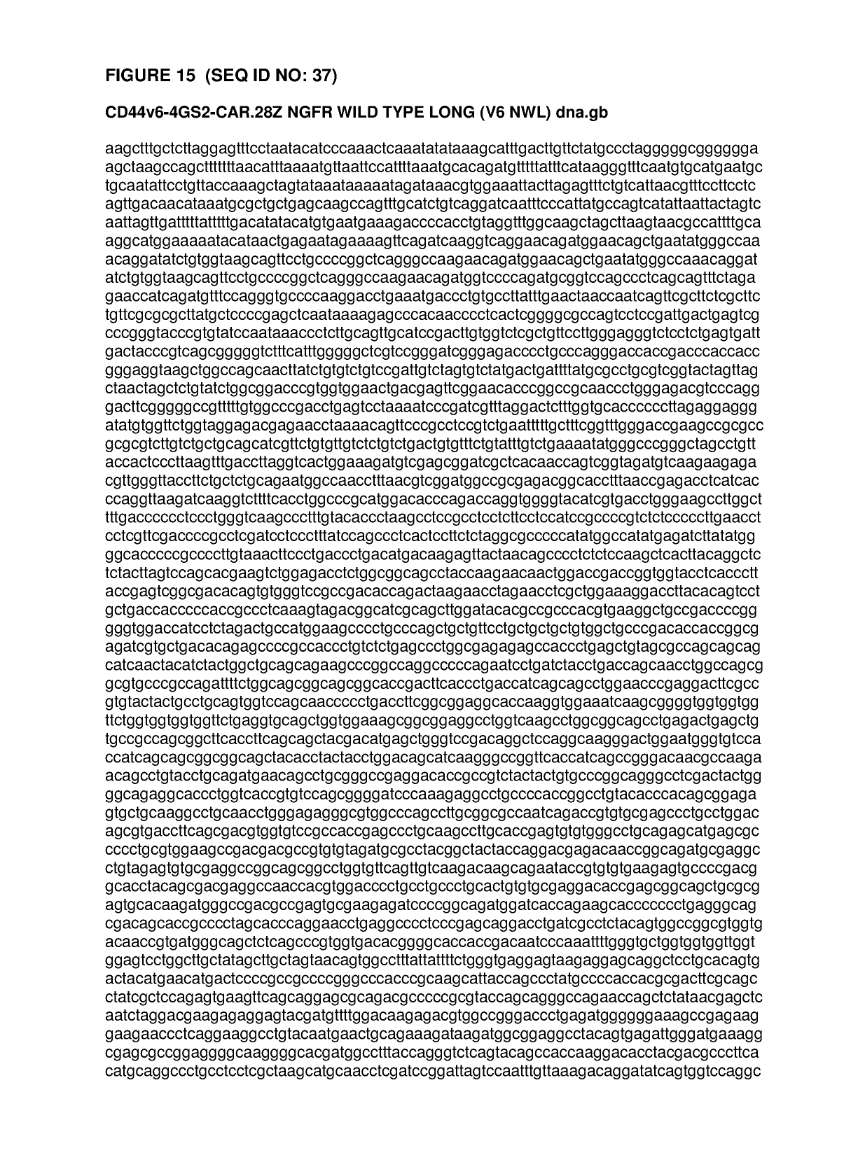

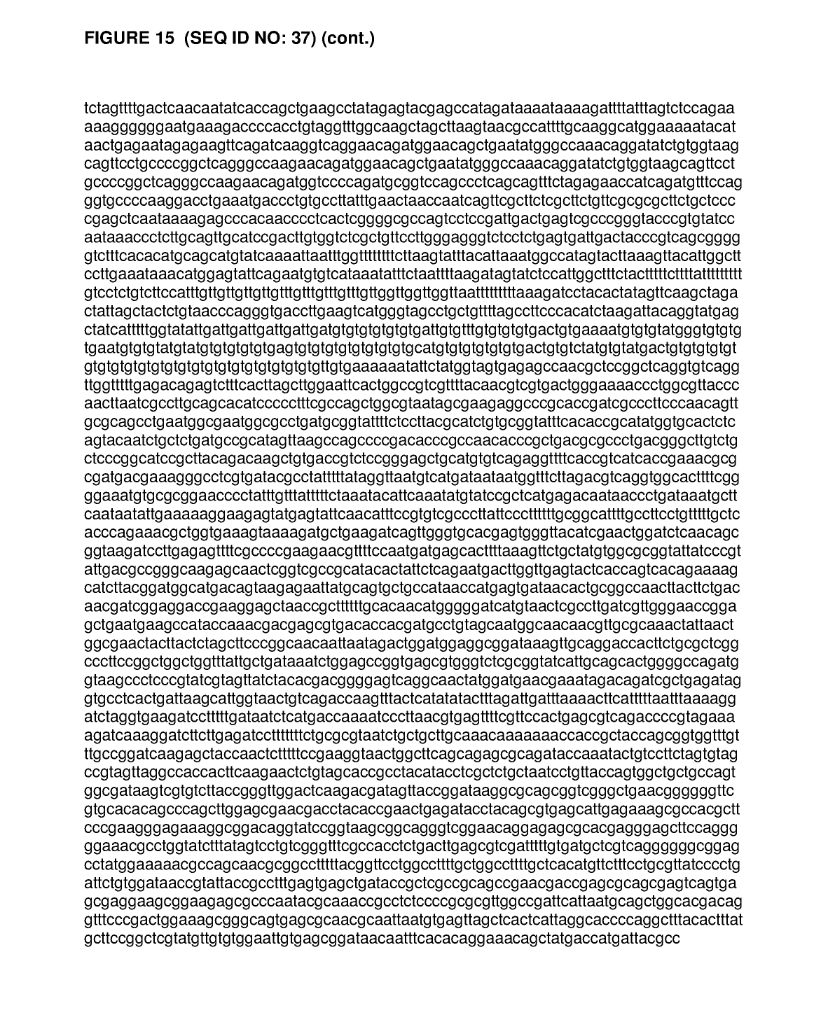

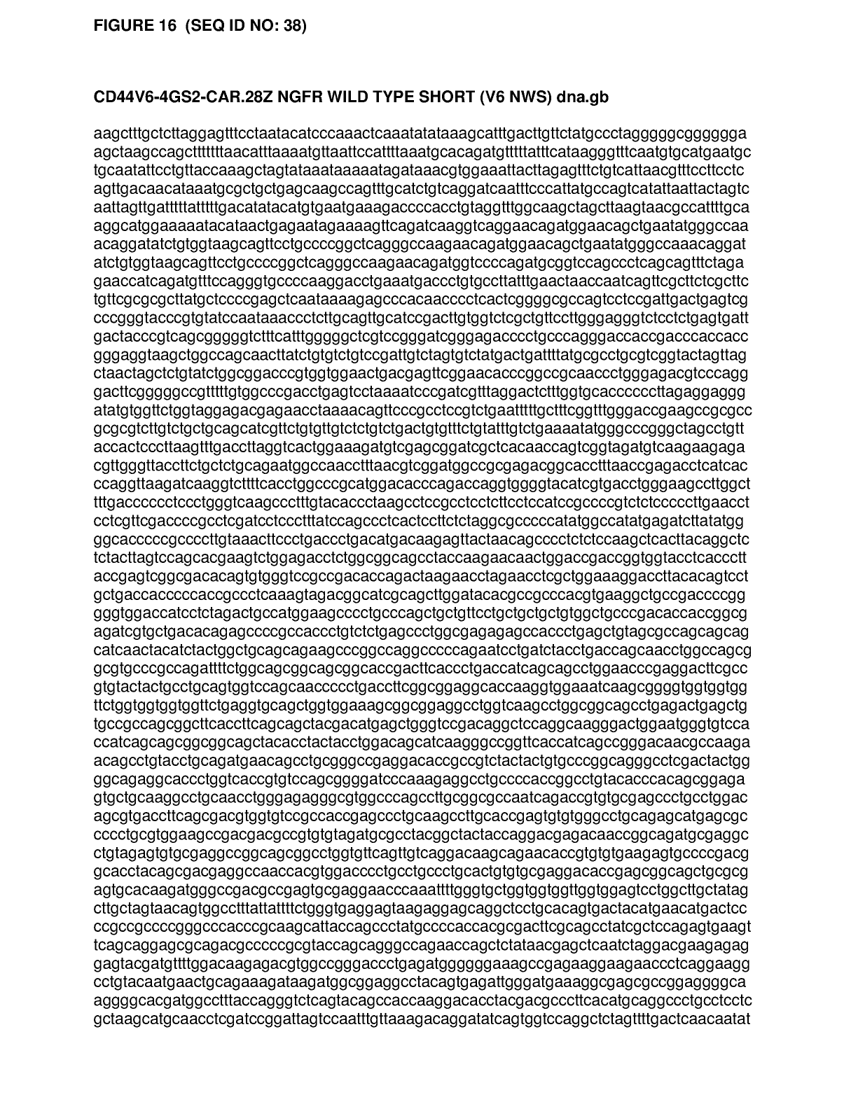

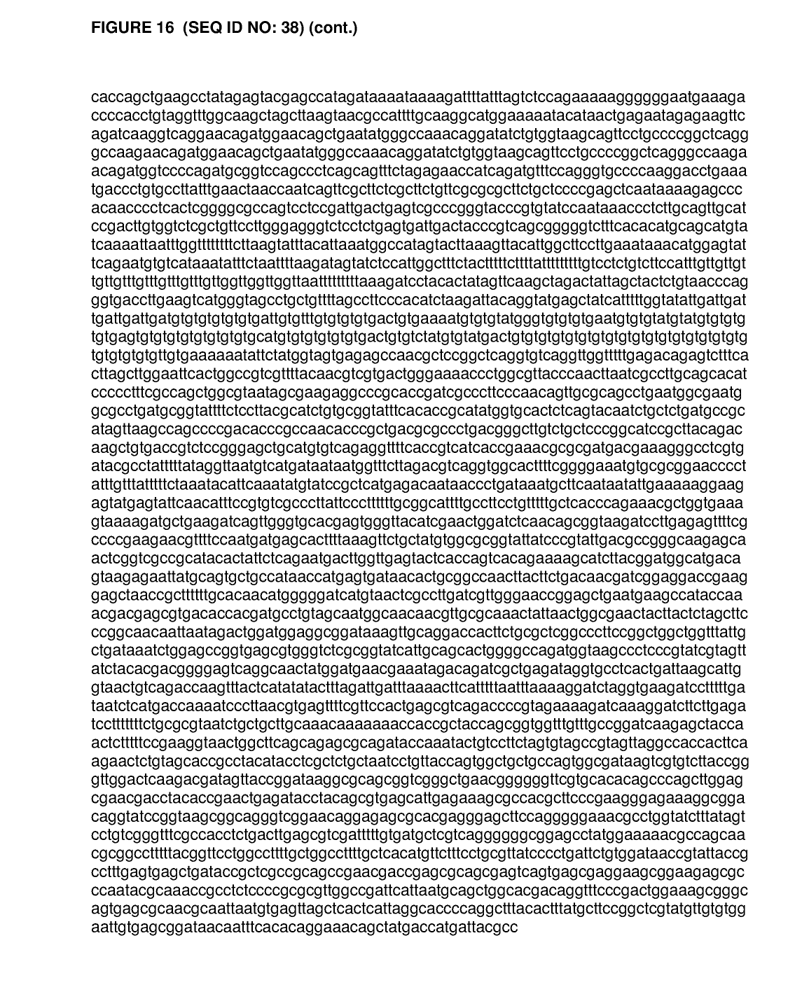

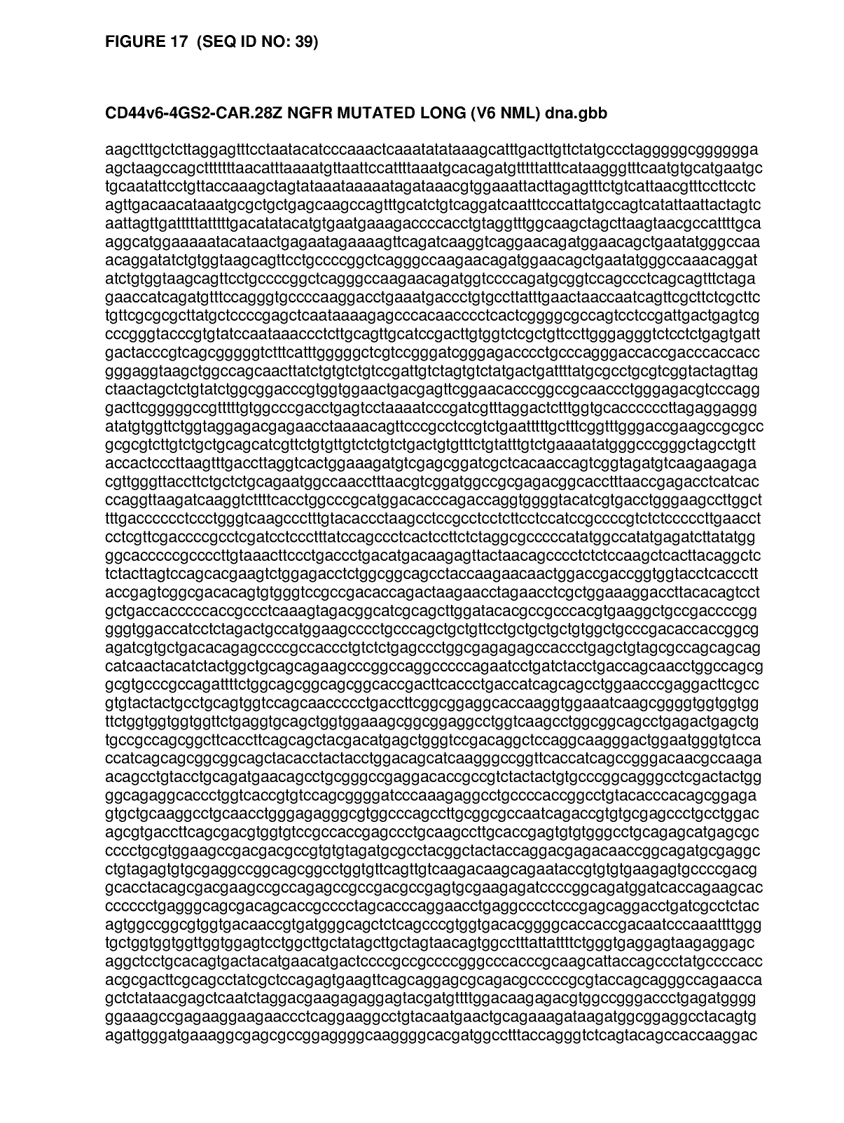

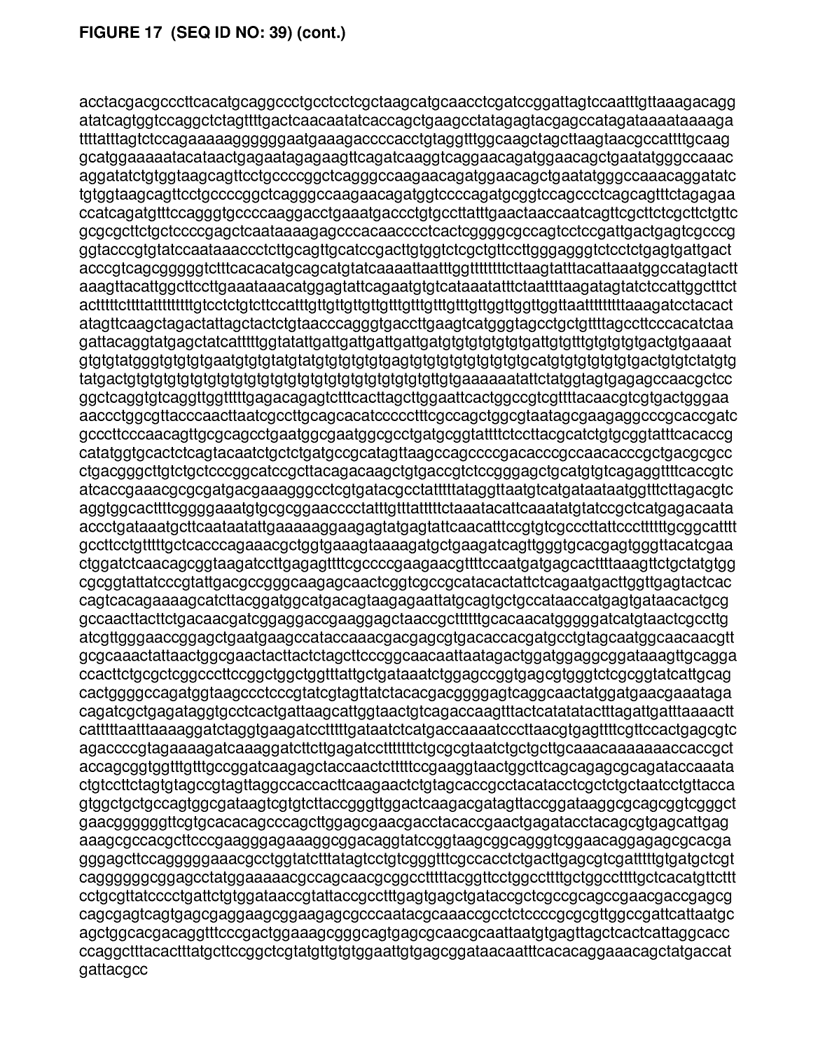

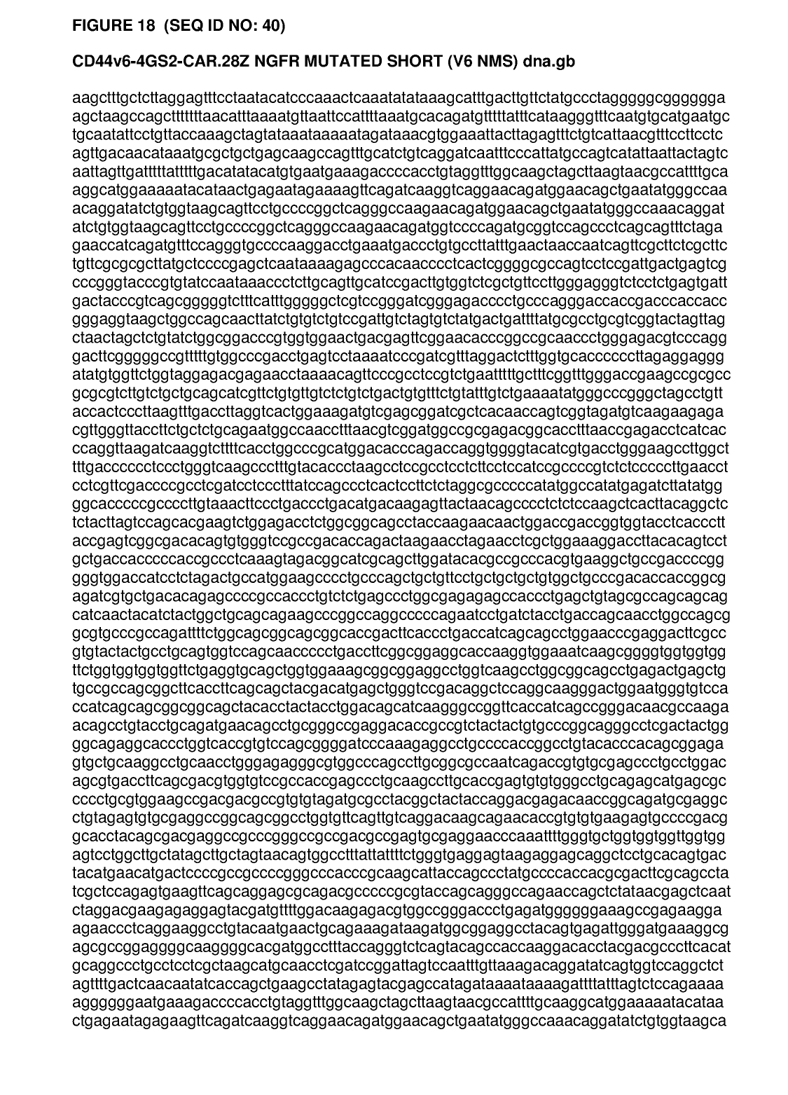

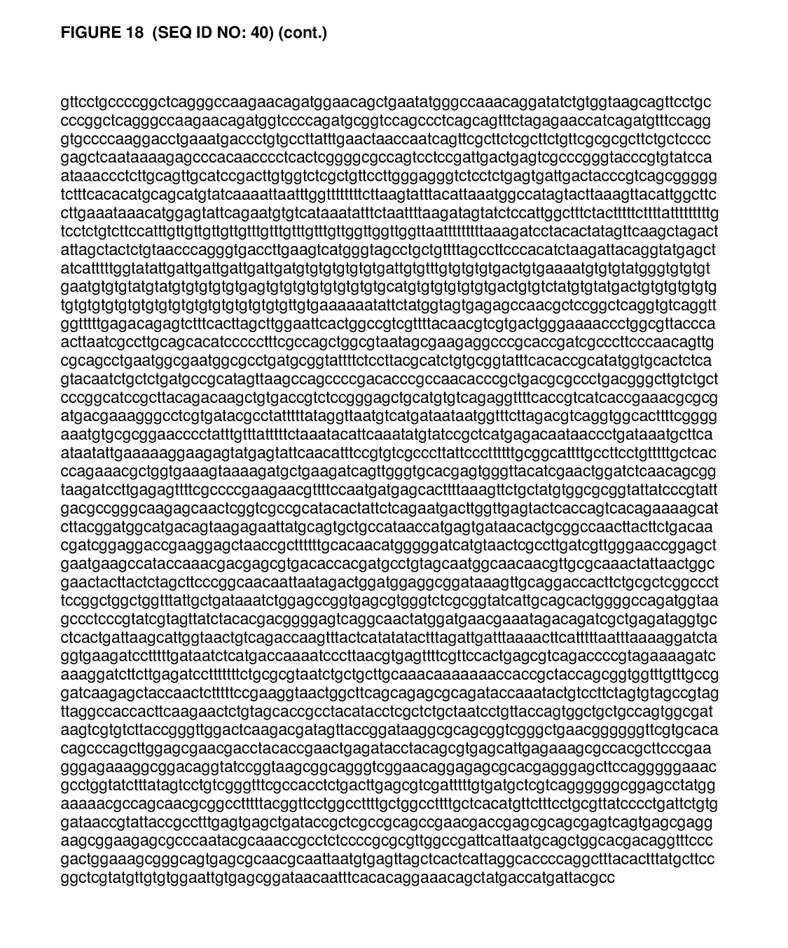

Spacers may be synthesised (e.g. by GENEART), flanked by specific restriction sites (e.g. BamH1 and PfIMI) to allow cloning into our original CD44v6-specific, second-generation CAR construct (FIG. 2; SEQ ID NO: 15) in place of the IgG1 CH2CH3 spacer. All constructs may be codon-optimised for expression in humans. All constructs may be expressed using SFG-RV backbones (a splicing MoMLV-based retroviral vector commonly used (Riviere et al. (1995) Proc. Natl. Acad. Sci. USA 92:6733-7)).

### Spacer LNGFR Wild-Type Long (NWL):

Protein sequence (SEQ ID NO: 1):
KEACPTGLYTHSGECCKACNLGEGVAQPCGANQTVCEPCLDSVTFSDVVS
ATEPCKPCTECVGLQSMSAPCVEADDAVCRCAYGYYQDETTGRCEACRVC
EAGSGLVFSCQDKQNTVCEECPDGTYSDEANHVDPCLPCTVCEDTERQLR
ECTRWADAECEEIPGRWITRSTPPEGSDSTAPSTQEPEAPPEQDLIASTV
AGVVTTVMGSSQPVVTRGTTDN
Nucleotide sequence (SEQ ID NO: 2):
AAAGAGGCCTGCCCCACCGGCCTGTACACCCACAGCGGAGAGTGCTGCA
AGGCCTGCAACCTGGGAGAGGGCGTGGCCCAGCCTTGCGGCGCCAATCA
GACCGTGTGCGAGCCCTGCCTGGACAGCGTGACCTTCAGCGACGTGGTG
TCCGCCACCGAGCCCTGCAAGCCTTGCACCGAGTGTGTGGGCCTGCAGA
GCATGAGCGCCCCCTGCGTGGAAGCCGACGACGCCGTGTGTAGATGCGC
CTACGGCTACTACCAGGACGAGACAACCGGCAGATGCGAGGCCTGTAGA
GTGTGCGAGGCCGGCAGCGGCCTGGTGTTCAGTTGTCAAGACAAGCAGA
ATACCGTGTGTGAAGAGTGCCCCGACGGCACCTACAGCGACGAGGCCAA
CCACGTGGACCCCTGCCTGCCCTGCACTGTGTGCGAGGACACCGAGCGG
CAGCTGCGCGAGTGCACAAGATGGGCCGACGCCGAGTGCGAAGAGATCC
CCGGCAGATGGATCACCAGAAGCACCCCCCCTGAGGGCAGCGACAGCAC
CGCCCCTAGCACCCAGGAACCTGAGGCCCCTCCCGAGCAGGACCTGATC
GCCTCTACAGTGGCCGGCGTGGTGACAACCGTGATGGGCAGCTCTCAGC
CCGTGGTGACACGGGGCACCACCGACAAT

### Spacer LNGFR Wild-Type Short (NWS):

Protein sequence (SEQ ID NO: 3):
KEACPTGLYTHSGECCKACNLGEGVAQPCGANQTVCEPCLDSVTFSDVVS
ATEPCKPCTECVGLQSMSAPCVEADDAVCRCAYGYYQDETTGRCEACRVC
EAGSGLVFSCQDKQNTVCEECPDGTYSDEANHVDPCLPCTVCEDTERQLR
ECTRWADAECEE
Nucleotide sequence (SEQ ID NO: 4):
AAAGAGGCCTGCCCCACCGGCCTGTACACCCACAGCGGAGAGTGCTGCAA
GGCCTGCAACCTGGGAGAGGGCGTGGCCCAGCCTTGCGGCGCCAATCAGA
CCGTGTGCGAGCCCTGCCTGGACAGCGTGACCTTCAGCGACGTGGTGTCC
GCCACCGAGCCCTGCAAGCCTTGCACCGAGTGTGTGGGCCTGCAGAGCAT
GAGCGCCCCCTGCGTGGAAGCCGACGACGCCGTGTGTAGATGCGCCTACG
GCTACTACCAGGACGAGACAACCGGCAGATGCGAGGCCTGTAGAGTGTGC
GAGGCCGGCAGCGGCCTGGTGTTCAGTTGTCAGGACAAGCAGAACACCGT
GTGTGAAGAGTGCCCCGACGGCACCTACAGCGACGAGGCCAACCACGTGG
ACCCCTGCCTGCCCTGCACTGTGTGCGAGGACACCGAGCGGCAGCTGCGC
GAGTGCACAAGATGGGCCGACGCCGAGTGCGAGGAA

### Spacer LNGFR Mutated Long (NML):

Protein sequence (SEQ ID NO: 5):
KEACPTGLYTHSGECCKACNLGEGVAQPCGANQTVCEPCLDSVTFSDVVS
ATEPCKPCTECVGLQSMSAPCVEADDAVCRCAYGYYQDETTGRCEACRVC
EAGSGLVFSCQDKQNTVCEECPDGTYSDEAARAADAECEEIPGRWITRST
PPEGSDSTAPSTQEPEAPPEQDLIASTVAGVVTTVMGSSQPVVTRGTTDN
Nucleotide sequence (SEQ ID NO: 6):
AAAGAGGCCTGCCCCACCGGCCTGTACACCCACAGCGGAGAGTGCTGCAA
GGCCTGCAACCTGGGAGAGGGCGTGGCCCAGCCTTGCGGCGCCAATCAGA
CCGTGTGCGAGCCCTGCCTGGACAGCGTGACCTTCAGCGACGTGGTGTCC
GCCACCGAGCCCTGCAAGCCTTGCACCGAGTGTGTGGGCCTGCAGAGCAT
GAGCGCCCCCTGCGTGGAAGCCGACGACGCCGTGTGTAGATGCGCCTACG
GCTACTACCAGGACGAGACAACCGGCAGATGCGAGGCCTGTAGAGTGTGC
GAGGCCGGCAGCGGCCTGGTGTTCAGTTGTCAAGACAAGCAGAATACCGT
GTGTGAAGAGTGCCCCGACGGCACCTACAGCGACGAAGCCGCCAGAGCCG
CCGACGCCGAGTGCGAAGAGATCCCCGGCAGATGGATCACCAGAAGCACC
CCCCCTGAGGGCAGCGACAGCACCGCCCCTAGCACCCAGGAACCTGAGGC
CCCTCCCGAGCAGGACCTGATCGCCTCTACAGTGGCCGGCGTGGTGACAA
CCGTGATGGGCAGCTCTCAGCCCGTGGTGACACGGGGCACCACCGACAAT

### Spacer LNGFR Mutated Short (NMS):

Protein sequence (SEQ ID NO: 7):
KEACPTGLYTHSGECCKACNLGEGVAQPCGANQTVCEPCLDSVTFSDVV
SATEPCKPCTECVGLQSMSAPCVEADDAVCRCAYGYYQDETTGRCEACR
VCEAGSGLVFSCQDKQNTVCEECPDGTYSDEAARAADAECEE
Nucleotide sequence (SEQ ID NO: 8):
AAAGAGGCCTGCCCCACCGGCCTGTACACCCACAGCGGAGAGTGCTGCA
AGGCCTGCAACCTGGGAGAGGGCGTGGCCCAGCCTTGCGGCGCCAATCA
GACCGTGTGCGAGCCCTGCCTGGACAGCGTGACCTTCAGCGACGTGGTG
TCCGCCACCGAGCCCTGCAAGCCTTGCACCGAGTGTGTGGGCCTGCAGA
GCATGAGCGCCCCCTGCGTGGAAGCCGACGACGCCGTGTGTAGATGCGC
CTACGGCTACTACCAGGACGAGACAACCGGCAGATGCGAGGCCTGTAGA
GTGTGCGAGGCCGGCAGCGGCCTGGTGTTCAGTTGTCAGGACAAGCAGA
ACACCGTGTGTGAAGAGTGCCCCGACGGCACCTACAGCGACGAGGCCGC
CCGGGCCGCCGACGCCGAGTGCGAGGAA

### Legend

Underlined: TNFR cysteine-rich domain number 1.
Bold: TNFR cysteine-rich domain number 2.
Bold and underlined: TNFR cysteine-rich domain number 3.
Italics: TNFR cysteine-rich domain number 4.
Italics and underlined: Serine/Threonine rich stalk.

All publications mentioned in the above specification are herein incorporated by reference.

Various modifications and variations of the described products and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. A pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), wherein the CAR comprises an antigen-specific targeting domain which targets a tumour antigen.

2. The pharmaceutical product of claim 1, wherein the pharmaceutical product is in the form of a pharmaceutical composition.

3. The pharmaceutical product of claim 1 or 2 for use in therapy.

4. A pharmaceutical product comprising (a) a conjugation product of TNF and a tumour- or tumour vasculature-targeting peptide comprising an NGR, DGR, isoDGR or RGD motif; and (b) a cell comprising a chimeric antigen receptor (CAR), as a combined preparation for simultaneous, sequential or separate use in therapy, wherein the CAR comprises an antigen-specific targeting domain which targets a tumour antigen.

5. The pharmaceutical product of claim 1, wherein the tumour- or tumour vasculature-targeting peptide is a ligand of the CD13 receptor.

6. The pharmaceutical product of claim 5, wherein the tumour- or tumour vasculature-targeting peptide is a peptide comprising an NGR motif.

7. The pharmaceutical product of claim 6, wherein the peptide comprising an NGR motif comprises the sequence XNGRX′ (SEQ ID NO: 44), wherein X is selected form the group consisting of L, V, A, C, G, Y, P, H, K, Q and I, and X′ is selected from the group consisting of C, G, H, L, E, T, Q, R, S and P.

8. The pharmaceutical product of claim 6, wherein the peptide comprising an NGR motif comprises a sequence selected from the group consisting of CNGRCVSGCAGRC (SEQ ID NO: 45), NGRAHA (SEQ ID NO: 46), GNGRG (SEQ ID NO: 47), CVLNGRMEC (SEQ ID NO: 48), CNGRC (SEQ ID NO: 49), GCNGRC (SEQ ID NO: 50), CNGRCG (SEQ ID NO: 51), LNGRE (SEQ ID NO: 52), YNGRT (SEQ ID NO: 53), LQCICTGNGRGEWKCE (SEQ ID NO: 54), LQCISTGNGRGEWKCE (SEQ ID NO: 55), CICTGNGRGEWKC (SEQ ID NO: 56), CISTGNGRGEWKC (SEQ ID NO: 57), MRCTCVGNGRGEWTCY (SEQ ID NO: 58), MRCTSVGNGRGEWTCY (SEQ ID NO: 59), CTCVGNGRGEWTC (SEQ ID NO: 60) and CTSVGNGRGEWTC (SEQ ID NO: 61).

9. The pharmaceutical product of claim 6, wherein the peptide comprising an NGR motif comprises the sequence CNGRCG or GCNGRC.

10. The pharmaceutical product of claim 1, wherein the tumour antigen is selected from the group consisting of CD44, CD19, CD20, CD22, CD23, CD123, CS-1, ROR1, mesothelin, c-Met, PSMA, Her2, GD-2, CEA, MAGE A3 TCR and combinations thereof.

11. The pharmaceutical product of claim 1, wherein the tumour antigen is isoform 6 of CD44 (CD44v6).

12. The pharmaceutical product of claim 1, wherein the CAR comprises an extracellular spacer comprising at least part of the extracellular domain of human low affinity nerve growth factor receptor (LNGFR) or a derivative thereof.

13. The pharmaceutical product of claim 1, wherein the cell is a lymphocyte, preferably a T cell or a natural killer cell.

14. The pharmaceutical product of claim 1, wherein the TNF is TNFα.

15. A method of treating cancer comprising administering (a) a conjugation product of TNF and a tumour- or tumour vasculature-targeting peptide comprising an NGR, DGR, isoDGR or RGD motif; and (b) a cell comprising a chimeric antigen receptor (CAR), to a subject simultaneously, sequentially or separately, wherein the CAR comprises an antigen-specific targeting domain which targets a tumour antigen.

16. The method of claim 15, wherein the cell comprising a CAR is administered to the subject about 1-4 hours after administration of the conjugation product of TNF and a tumour- or tumour vasculature-targeting peptide.

17. The method of claim 15, wherein the cell comprising a CAR is administered to the subject more than about 12 hours after administration of the conjugation product of TNF and a tumour- or tumour vasculature-targeting peptide.

18. The method of claim 15, wherein the conjugation product of TNF and a tumour- or tumour vasculature-targeting peptide is administered to the subject in 3 doses over about a 1 week period.

19. The method of claim 15, wherein the tumour- or tumour vasculature-targeting peptide is a ligand of the CD13 receptor.

20. The method of claim 19, wherein the tumour- or tumour vasculature-targeting peptide is a peptide comprising an NGR motif.

21. The method of claim 20, wherein the peptide comprising an NGR motif comprises the sequence XNGRX′ (SEQ ID NO: 44), wherein X is selected form the group consisting of L, V, A, C, G, Y, P, H, K, Q and I, and X′ is selected from the group consisting of C, G, H, L, E, T, Q, R, S and P.

22. The method of claim 20 or 21, wherein the peptide comprising an NGR motif comprises a sequence selected from the group consisting of CNGRCVSGCAGRC (SEQ ID NO: 45), NGRAHA (SEQ ID NO: 46), GNGRG (SEQ ID NO: 47), CVLNGRMEC (SEQ ID NO: 48), CNGRC (SEQ ID NO: 49), GCNGRC (SEQ ID NO: 50), CNGRCG (SEQ ID NO: 51), LNGRE (SEQ ID NO: 52), YNGRT (SEQ ID NO: LQCICTGNGRGEWKCE (SEQ ID NO: 54), LQCISTGNGRGEWKCE (SEQ ID NO: 55), CICTGNGRGEWKC (SEQ ID NO: 56), CISTGNGRGEWKC (SEQ ID NO: 57), MRCTCVGNGRGEWTCY (SEQ ID NO: 58), MRCTSVGNGRGEWTCY (SEQ ID NO: 59), CTCVGNGRGEWTC (SEQ ID NO: 60) and CTSVGNGRGEWTC (SEQ ID NO: 61).

23. The method of claim 20, wherein the peptide comprising an NGR motif comprises the sequence CNGRCG (SEQ ID NO: 51) or GCNGRC (SEQ ID NO: 50).

24. The method of claim 15, wherein the tumour antigen is selected from the group consisting of CD44, CD19, CD20, CD22, CD23, CD123, CS-1, ROR1, mesothelin, c-Met, PSMA, Her2, GD-2, CEA, MAGE A3 TCR and combinations thereof.

25. The method of claim 15, wherein the tumour antigen is isoform 6 of CD44 (CD44v6).

26. The method of claim 15, wherein the CAR comprises an extracellular spacer comprising at least part of the extracellular domain of human low affinity nerve growth factor receptor (LNGFR) or a derivative thereof.

27. The method of claim 15, wherein the cell is a lymphocyte, preferably a T cell or a natural killer cell.

28. The method of claim 15, wherein the TNF is TNFα.
